(19) United States
(12) Patent Application Publication
(10) Pub. No.: US 2012/0271111 A1
(43) Pub. Date: Oct. 25, 2012

(54) BEAM SPLITTER APPARATUS, LIGHT SOURCE APPARATUS, AND SCANNING OBSERVATION APPARATUS

(75) Inventors: Mitsuru NAMIKI, Tokyo (JP); Yohei TANIKAWA, Tokyo (JP); Yasunobu IGA, Tokyo (JP); Shintaro TAKAHASHI, Tokyo (JP)

(73) Assignee: OLYMPUS CORPORATION, Tokyo (JP)

(21) Appl. No.: 13/461,096

(22) Filed: May 1, 2012

Related U.S. Application Data
(63) Continuation of application No. PCT/JP2010/055496, filed on Mar. 23, 2010.

(30) Foreign Application Priority Data
Nov. 2, 2009 (JP) 2009-251859

(51) Int. Cl. G02B 27/10; G01J 1/16; A61B 1/06; G02B 26/10

(52) U.S. Cl. 600/163; 359/618; 359/197.1; 250/201.1; 359/434; 359/205.1

(57) ABSTRACT

While one beam is being branched into a plurality of beams with different optical path lengths, the beams can be converged on the same position in the optical-axis direction with a simple structure even when relative angles between the beams differ. Provided is a beam splitter apparatus including a beam splitter that branches an input pulsed beam into two optical paths with different optical path lengths; relay optical systems that are disposed in the respective branching optical paths and that relay pupils in the optical paths; a beam splitter that multiplexes the relayed pulsed beams in the two optical paths; and a reflection optical system that endows pulsed beams branching off via the beam splitter with a relative angle.

### CROSS-REFERENCE TO RELATED APPLICATIONS

This application is a continuation application of PCT/JP2010/055496, filed on Mar. 23, 2010, the contents of which are incorporated herein by reference.

This application is based on Japanese Patent Application No. 2009-251859, filed on Nov. 2, 2009, the contents of which are incorporated herein by reference.

### TECHNICAL FIELD

The present invention relates to beam splitter apparatuses, light source apparatuses, and scanning observation apparatuses.

### BACKGROUND ART

Beam splitter apparatuses for branching one laser beam emitted from a light source into a plurality of laser beams are well known (refer to, for example, Patent Literature 1). This kind of beam splitter apparatus includes at least two highly reflecting mirrors that are disposed at mutually different distances from a flat semi-transparent mirror interposed therebetween and is provided with a portion formed as a total reflector or an anti-reflection member on the semi-transparent mirror.

According to this beam splitter apparatus, a laser beam entering from one side of the semi-transparent mirror is branched by the semi-transparent mirror, reflected by highly reflecting mirrors disposed on either side of the semi-transparent mirror, and returns to the semi-transparent mirror. Through repetition of this step, one laser beam is branched into a plurality of laser beams with different optical path lengths. The plurality of resultant laser beams can be converged on one position by endowing the highly reflecting mirrors with a minute angle.

### CITATION LIST

### Patent Literature

{PTL 1}

Japanese Patent No. 3927513

### SUMMARY OF INVENTION

### Technical Problem

However, when the beam splitter apparatus disclosed in Patent Literature 1 is to be applied to a scanning observation apparatus, such as a scanning microscope, it is necessary to not only effectively produce optical responses from the subject but also detect those optical responses by differentiating them for each radiation position.

More specifically, when the subject is to be irradiated with a plurality of light beams, as with the beam splitter apparatus described in Patent Literature 1, optical responses produced at different radiation positions spatially overlap one another on the detector due to scattering of light on the surface and in the interior of the subject, and these optical responses cannot be differentiated for each radiation position. The deeper the positions in the subject from which optical responses are to be observed, the more intense the scattering of light and the more noticeable this spatial overlapping. In addition, light beams to be radiated on the subject needs to be adjusted to have appropriate intervals. However, with the beam splitter apparatus disclosed in Patent Literature 1, the point of convergence shifts in the optical-axis direction when the branching laser beams are to be set at different relative angles merely by angle setting of the highly reflecting mirrors. Angle setting alone of the highly reflecting mirrors is not satisfactory to endow the laser beams with different relative angles without shifting the point of convergence in the optical-axis direction, but rather, their positions also need to be shifted. Furthermore, when a laser beam is branched into a plurality of laser beams, fine angle setting of the reflecting mirrors is required for each beam branch. For this reason, the work of setting the highly reflecting mirrors is intricate, and the structure of the apparatus also becomes complicated.

The present invention is to provide a beam splitter apparatus and a light source apparatus that can detect the responses in the subject, resulting from irradiation with a plurality of light beams, by separating them on the time axis, even if the responses spatially overlapping one another on the detector, as well as providing a scanning observation apparatus capable of fast scanning using this beam splitter apparatus. Furthermore, the present invention is to provide a beam splitter apparatus and a light source apparatus that can branch one beam into a plurality of beams with different optical path lengths and, at the same time, can converge, with a simple structure, those laser beams on the same position in the optical-axis direction, despite the different relative angles between the beams, as well as providing a scanning observation apparatus capable of fast scanning using this beam splitter apparatus.

### Solution to Problem

A first aspect according to the present invention is a beam splitter apparatus that generates a plurality of pulsed beams to be radiated on a subject from an input pulsed beam, and the beam splitter apparatus includes at least one branching section that branches the input pulsed beam into two optical paths; at least one delaying section that endows pulsed beams passing along the two optical paths branching off via the branching section with a relative time delay to sufficiently separate responses in the subject caused by the pulsed beam; and a beam-angle setting section that endows the plurality of pulsed beams, endowed with the relative time delay by the delaying section, with a relative angle and converges the plurality of pulsed beams on the same position.

According to the first aspect of the present invention, the input pulsed beam is branched into the two optical paths by the branching section. The pulsed beam that has branched into each of the optical paths is endowed with the relative time delay by the delaying section while passing along each of the optical paths. Then, the two pulsed beams, endowed with the relative time delay, are endowed with the relative angle by the beam-angle setting section, converged on the same position, and radiated on the subject.

Because the pulsed beams are converged on the same position with the relative angle therebetween, all the pulsed beams can be transmitted by arranging the position of convergence of the pulsed beams at a pupil position of an optical system (e.g., an objective optical system) downstream thereof or a position that is optically conjugate to it. Then, the pulsed beams can be focused at a focal position of the optical system and spatially spaced apart in the form of multiple points.

In this case, the relative time delay caused by the delaying section is longer than the time of the response such as fluorescence or scattering in the subject. Then, the responses in the subject resulting from the pulsed beams are prevented from being mixed and can be detected by separating them on the time axis.

In the above-described aspect, a relay optical system that is disposed in each of the optical paths branching off via the branching section and that relays a pupil in each of the optical paths; and at least one multiplexing section that multiplexes the plurality of pulsed beams relayed by the relay optical systems may be provided. The beam-angle setting section may endow one of the pulsed beams branching off via the branching section with an angle so as to have a relative angle with respect to the other pulsed beam.

By doing so, the input pulsed beam is branched by the branching section into the two optical paths with different optical path lengths, and the pulsed beams are relayed by the relay optical systems disposed in the respective optical paths and are multiplexed by the multiplexing section. At this time, one of the pulsed beams branching into the two optical paths via the branching section is endowed with an angle by the beam-angle setting section so as to have a relative angle with respect to the other pulsed beam. By doing so, the pulsed beams in the two optical paths having different optical path lengths and endowed with the relative angle can be converged on one position.

In this case, because the pupils of the pulsed beams branching into the two optical paths via the branching section are relayed by the relay optical systems disposed in the respective optical paths, the point of convergence of the pulsed beams can be prevented from being shifted in an optical-axis direction even when the branching pulsed beams are set to different relative angles. In short, according to this aspect, even when the relative angles of the pulsed beams are different, the plurality of pulsed beams can be converged on the same pupil position in the optical-axis direction with a simple structure in the form of the relay optical systems.

As a result, even when relative angles of the pulsed beams are changed, the pulsed beams can be made incident on the optical systems disposed downstream thereof under the same incidence conditions. For example, by converging a plurality of pulsed beams endowed with a relative angle on the pupil position of a microscope objective lens, the pulsed beams can be radiated at different positions on the focal plane of the objective lens. The intervals of the radiation positions can be changed by making the relative angles different, and the amount of light can be prevented from fluctuating at this time.

In the above-described aspect, the relay optical system may include at least one pair of lenses, and the beam-angle setting section may be disposed between the one pair of lenses or between a plurality of pairs of lenses.

By doing so, the pupil is relayed by the one pair of lenses even when the branching pulsed beams are endowed with a relative angle by the beam-angle setting section, and the point of convergence of the pulsed beams can be prevented from being shifted in the optical-axis direction. Furthermore, as a result of a plurality of pairs of such lenses being provided and the pupils in the two optical paths being relayed by the plurality of pairs of theses lenses, the lens diameter can be reduced.

In the above-described aspect, the beam-angle setting section may include a first mirror that reflects a pulsed beam branching off via the branching section; a second mirror that reflects the pulsed beam, reflected by the first mirror, towards the multiplexing section; and a rectilinear translation mechanism that rectilinearly translates the first mirror and the second mirror together in the optical-axis direction therebetween.

A pulsed beam branching off via the branching section can be endowed with a relative angle by parallel moving the first mirror and the second mirror together by means of the rectilinear translation mechanism in the optical-axis direction between these mirrors.

In the above-described aspect, the beam-angle setting section may include a mirror that reflects the pulsed beams branching off via the branching section towards the multiplexing section and a swing mechanism that swings the mirror about an axis orthogonal to optical axes of the pulsed beams.

The pulsed beams branching off via the branching section can be endowed with a relative angle by swinging the mirror, with the swing mechanism, about an axis orthogonal to the optical axes of the pulsed beams.

In the above-described aspect, the beam-angle setting section may include a swing mechanism that swings at least one of the branching section and the multiplexing section about an axis orthogonal to optical axes of the pulsed beams.

The pulsed beams branching off via the branching section can be endowed with a relative angle by swinging at least one of the branching section and the multiplexing section, with the swing mechanism, about an axis orthogonal to optical axes of the pulsed beams.

In the above-described aspect, a plurality of units in series that each include the branching section, the multiplexing section, the relay optical systems, and the beam-angle setting section may be provided, and the beam-angle setting sections may be disposed between the respective branching sections and the respective multiplexing sections.

The input pulsed beam can be branched into a plurality of optical paths, and each of the branching pulsed beams can be endowed with a relative angle by the beam-angle setting section by providing a plurality of units in series that include the branching section, the multiplexing section, the relay optical systems, and the beam-angle setting section. As a result, pulsed beams in a plurality of optical paths, having different optical path lengths and endowed with a relative angle, can be converged on one position.

In the above-described aspect, at least one multiplexing/branching section that multiplexes the pulsed beams in the two optical paths branching off via the branching section and that branches the multiplexed pulsed beams into two optical paths with different optical path lengths may be provided. The relay optical system may be disposed in each of the optical paths branching off via the branching/multiplexing section, and the beam-angle setting section may endow pulsed beams branching off via the multiplexing/branching section with a relative angle.

As a result of the at least one multiplexing/branching section being provided, the input pulsed beam can be branched into a plurality of optical paths by the branching section and the multiplexing/branching section, and each of the branching pulsed beams can be endowed with a relative angle by the beam-angle setting section. As a result, pulsed beams in a plurality of optical paths, having different optical path lengths and endowed with a relative angle, can be converged on one position.

In the above-described aspect, a polarization modulator that is disposed in one of the optical paths upstream of the multiplexing section and that makes the polarization states of the two optical paths orthogonal to each other may be provided. The multiplexing section may be a polarizing beam splitter.

One of the pulsed beams in the two optical paths branching off via the branching section or the multiplexing/branching section can be transmitted, while the other is reflected, by enabling the polarization modulator to make the polarization states of the two optical paths orthogonal to each other and forming the multiplexing section of the polarizing beam splitter. As a result, all the pulsed beams in the two optical paths can be multiplexed by the multiplexing section, thus suppressing the amount of light loss of these pulsed beams, thereby increasing the utilization efficiency of the input pulsed beam.

Furthermore, a second aspect according to the present invention is a beam splitter apparatus that generates a plurality of pulsed beams to be radiated on a subject from an input pulsed beam, and the beam splitter apparatus includes at least one branching section that branches the input pulsed beam into two optical paths; at least one delaying section that endows pulsed beams passing along the two optical paths branching off via the branching section with a relative time delay to sufficiently separate responses in the subject caused by the pulsed beams; at least one multiplexing section that multiplexes the two pulsed beams endowed with the time delay by the delaying section; a stationary displacing section that is disposed in each of the optical paths branching off via the branching section, causes pulsed beams multiplexed by the multiplexing section to be incident on different positions of the multiplexing section, and makes principal rays of the pulsed beams parallel to one another after the last multiplexing section; and at least one lens disposed after the last multiplexing section.

According to this aspect, the input pulsed beam is branched by the branching section into the two optical paths. The pulsed beam that has branched into each of the optical paths is endowed with the relative time delay by the delaying section while passing along each of the optical paths. Then, the two pulsed beams endowed with the relative time delay are subjected to adjustment of their incident positions on the multiplexing section by the stationary displacing sections provided in the optical paths and are then multiplexed by the multiplexing section. Principal rays of the pulsed beams are adjusted to be parallel to each other by the stationary displacing sections after the last multiplexing section, and the pulsed beams are correctly converged on the same position by the lens disposed downstream thereof.

In this case, because the delaying section endows the two pulsed beams with the relative time delay to sufficiently separate the responses in the subject, the responses in the subject resulting from the pulsed beams are prevented from being mixed and can be detected by separating them on the time axis.

In the above-described aspect, a relay optical system that is disposed in each of the optical paths branching off via the branching section and that relays a pupil in each of the optical paths may be provided.

By doing so, the beam diameters of the pulsed beams branching off via the branching section can be made the same by the relay optical systems. As a result, when a plurality of the generated pulsed beams is applied to a scanning observation apparatus, the resolving power can be prevented from changing.

Furthermore, in the above-described aspect, the stationary displacing sections may include at least two mirrors and a rectilinear translation mechanism that rectilinearly translates at least one of the mirrors in a plane parallel to an optical axis of a pulsed beam incident on the mirror so as to change an optical path length between the mirrors.

The optical path length between the mirrors can be changed by the operation of the rectilinear translation mechanism, thereby changing the intervals of the incident positions, on the multiplexing section, of the two pulsed beams multiplexed by the multiplexing section.

Furthermore, in the above-described aspect, the rectilinear translation mechanism may move the two mirrors in a direction parallel to an optical axis between the mirrors.

By doing so, the intervals of the incident positions, on the multiplexing section, of the two pulsed beams multiplexed by the multiplexing section can be changed, and the optical path length can be prevented from changing even in that case. As a result of the optical path length being prevented from changing, it is not necessary to set the optical path length anew. If the pulsed beam is a laser beam, it diverges at a predetermined angle depending on the beam diameter while propagating. Because of this, the beam diameter after propagating changes if the optical path length changes. As a result of the optical path length being prevented from changing, the beam diameter can be prevented from changing, thereby preventing the resolving power from changing when this aspect is applied to a scanning observation apparatus.

Furthermore, in the above-described aspect, at least one lens group and a lens-group moving mechanism that moves the lens group in a direction orthogonal to the optical axis by the same amount as an amount of displacement of the optical axis in synchronization with displacement of the optical axis by the stationary displacing section may be provided downstream of the stationary displacing sections.

By doing so, even when the optical axis is displaced by the stationary displacing sections, the lens group can be moved by the lens-group moving mechanism in a direction orthogonal to the optical axis by the same amount as the amount of displacement of the optical axis. As a result, even when the relative angle of the pulsed beams is changed by the stationary displacing sections, the principal rays of the pulsed beams after being multiplexed can be kept parallel to one another, thereby preventing the point of convergence from shifting in the optical-axis direction.

Furthermore, between downstream of the above-described stationary displacing section and at least one lens disposed after the above-described last multiplexing section, at least one pair of lenses (36b:104c and 37b:105a) may be disposed such that the focal positions of the lenses coincide with one another, as shown in FIG. 19 (in short, they serve as a 4f optical system).

By doing so, even when the optical axis is displaced by the stationary displacing section, because an optical system downstream thereof serves as a 4f optical system, the principal rays of pulsed light beams after the last multiplexing section can be kept parallel to one another, thereby preventing the point of convergence from shifting in the optical-axis direction.

Furthermore, a third aspect according to the present invention is a beam splitter apparatus that generates a plurality of pulsed beams radiated on a subject from an input pulsed beam, and the beam splitter apparatus includes at least one branching section that branches the input pulsed beam into two; at least two light-guide members with different optical path lengths that propagate the pulsed beams branching off via the branching section; and a beam-angle setting section that endows a plurality of pulsed beams emitted from exit ends of the plurality of light-guide members with a relative angle and that converges the plurality of pulsed beams on the same position.

According to the above-described aspect, the input pulsed beam is branched into two by the branching section, and the branching pulsed beams propagate along the at least two light-guide members, are emitted from the exit ends of the light-guide members, are endowed with a relative angle by the beam-angle setting section, and are converged on the same position. Because the at least two light-guide members have optical path lengths different from one another, the pulsed beams emitted from the exit ends are endowed with a relative time delay. As a result, the pulsed beams can be endowed with a sufficient time delay merely by adjusting the length of light-guide members, without increasing the size of the apparatus, and the responses in the subject resulting from the pulsed beams can be prevented from being mixed and can be detected by separating them on the time axis.

In this case, the beam-angle setting section may be constructed by setting the directions of the exit ends such that the optical axes of the light-guide members intersect one another at one point. Alternatively, if the light-guide members are set such that the optical axes are parallel, the beam-angle setting section may be in the form of a lens that converges the pulsed beams emitted from these exit ends on the same position.

Furthermore, a fourth aspect according to the present invention is a light source apparatus including a pulsed light source that emits a pulsed beam; and one of the above-described beam splitter apparatuses that receives the pulsed beam emitted from the pulsed light source.

According to this light source apparatus, a bundle of a plurality of pulsed beams emitted from the pulsed light source, having different optical path lengths and endowed with a relative angle, can be converged on the same position and can all be made to pass through the pupil position of an optical system disposed downstream thereof.

In the above-described aspect, a scanning section that spatially scans a plurality of pulsed beams emitted from the beam splitter apparatus may be provided.

By doing so, while forming many spots on the subject, a plurality of pulsed beams endowed with a time delay can be scanned over these spots on the subject through the operation of the scanning section. As a result, a wider range of the subject can be irradiated with pulsed beams.

Furthermore, a fifth aspect according to the present invention is a light source apparatus including a pulsed light source that emits a pulsed beam; one of the above-described beam splitter apparatuses that receives the pulsed beam emitted from the pulsed light source; and a scanning section that spatially scans a plurality of pulsed beams emitted from the beam splitter apparatus by spatially vibrating the exit ends of the plurality of light-guide members.

A sixth aspect according to the present invention is a scanning observation apparatus including one of the above-described beam splitter apparatuses; a scanning section that scans a plurality of pulsed beams from the beam splitter apparatus over the subject; an observation optical system that radiates the pulsed beams scanned by the scanning section on the subject; and a detecting section that detects the signal light from the subject.

In the above-described aspect, a processing section that synchronizes the signal light detected by the detecting section with the scanned pulsed beams; a restoring section that reconstructs the signal light synchronized by the processing section as two-dimensional information or three-dimensional information in association with sites on the subject; and a display section that displays the two-dimensional information or three-dimensional information may be provided.

According to this scanning observation apparatus, a plurality of pulsed beams having different optical path lengths and endowed with a relative angle can be converged on one position by the beam splitter apparatus and radiated on different positions of the subject. Then, an image of the subject can be generated by scanning radiation positions on the subject two-dimensionally or three-dimensionally with the scanning section and detecting light from the subject with the detecting section.

### ADVANTAGEOUS EFFECTS OF INVENTION

The present invention affords an advantage in that beams can be converged on the same position in the optical-axis direction with a simple structure, even if relative angles between the beams differ.

### BRIEF DESCRIPTION OF DRAWINGS

FIG. 1 is a schematic structural diagram of a beam splitter apparatus of a first embodiment according to the present invention.

FIG. 2 is a diagram depicting temporal multiplexing by the beam splitter apparatus of FIG. 1, where (a) shows a time delay produced in a reflection optical system and (b) shows an optical pulse train.

FIG. 3 is a schematic structural diagram of a beam splitter apparatus according to a modification of FIG. 1.

FIG. 4 is a schematic structural diagram of a beam splitter apparatus presented as a reference embodiment according to the present invention.

FIG. 5 is a diagram depicting temporal multiplexing by the beam splitter apparatus of FIG. 4, where (a) shows a time delay produced by a reflection optical system, (b) shows time delays produced by a reflection optical system, and (c) shows an optical pulse train.

FIG. 6 is a schematic structural diagram of a beam splitter apparatus of a second embodiment according to the present invention.

FIG. 7 is a diagram depicting a method of deflecting a pulsed beam with the beam splitter apparatus of FIG. 6, where (a) shows a case where no deflection is performed and (b) shows a case where deflection is performed.

FIG. 8 is a schematic structural diagram of a beam splitter apparatus of a modification of FIG. 6.

FIG. 9 is a schematic structural diagram of a beam splitter apparatus of a third embodiment according to the present invention.

FIG. 10 is a schematic structural diagram of a beam splitter apparatus of a fourth embodiment according to the present invention.

FIG. 11 is a schematic structural diagram of a beam splitter apparatus of a modification of FIG. 10.

FIG. 12 is a schematic structural diagram of a beam splitter apparatus of a fifth embodiment according to the present invention.

FIG. 13 is a schematic structural diagram of a beam splitter apparatus of a sixth embodiment according to the present invention.

FIG. 14 is a schematic structural diagram of a scanning microscope of a seventh embodiment according to the present invention.

FIG. 15 is a diagram depicting temporal multiplexing by the scanning microscope of FIG. 14, where (a) shows a pulse train of pulsed beams and (b) shows a pulse train of detected fluorescence.

FIG. 16 is a schematic structural diagram depicting a beam splitter apparatus of an eighth embodiment according to the present invention.

FIG. 17 is a schematic structural diagram depicting a beam splitter apparatus of a ninth embodiment according to the present invention.

FIG. 18 is a schematic structural diagram depicting a beam splitter apparatus of a tenth embodiment according to the present invention.

FIG. 19 is a schematic structural diagram depicting a beam splitter apparatus of an eleventh embodiment according to the present invention.

FIG. 20 is a magnified view of area AA of FIG. 19.

FIG. 21 is a magnified view of area AB of FIG. 19.

FIG. 22 is a schematic structural diagram depicting a beam splitter apparatus of a twelfth embodiment according to the present invention.

FIG. 23 is a diagram depicting paths with optical path lengths of the beam splitter apparatus of FIG. 22, where (a) shows a path with the smallest optical path length, (b) shows a path with the second smallest optical path length, (c) shows a path with the second largest optical path length, and (d) shows a path with the largest optical path length in a solid line.

FIG. 24 is a diagram depicting the time intervals of four pulsed beams generated by the beam splitter apparatus of FIG. 22.

FIG. 25 is a diagram depicting the relationship between the intervals of the pulsed beams of FIG. 24 and coherence time.

FIG. 26 is a schematic structural diagram depicting a modification of the application example of the beam splitter apparatus in FIG. 22.

FIG. 27 is an overall structural diagram depicting one example of a fluoroscopy apparatus using the beam splitter apparatus of FIG. 23.

FIG. 28 is a diagram depicting the relationship between pulsed beams radiated on a subject by the fluoroscopy apparatus of FIG. 27 and fluorescence emitted from the subject.

FIG. 29 is a schematic structural diagram depicting a beam splitter apparatus of a thirteenth embodiment according to the present invention.

FIG. 30 is a diagram depicting a cross-sectional view of an optical fiber bundle of four optical fibers of the beam splitter apparatus of FIG. 29.

FIG. 31 is a cross-sectional view depicting one exemplary morphology of the end of an optical fiber bundle having four cores arranged in a square in a fused and integrated cladding, instead of bundling the four optical fibers of FIG. 30.

FIG. 32 is a cross-sectional view of a modification of the arrangement of the cores in FIG. 31.

FIG. 33 is an overall structural diagram depicting one example of a fluoroscopy apparatus provided with the beam splitter apparatus of FIG. 29.

### DESCRIPTION OF EMBODIMENTS

### First Embodiment

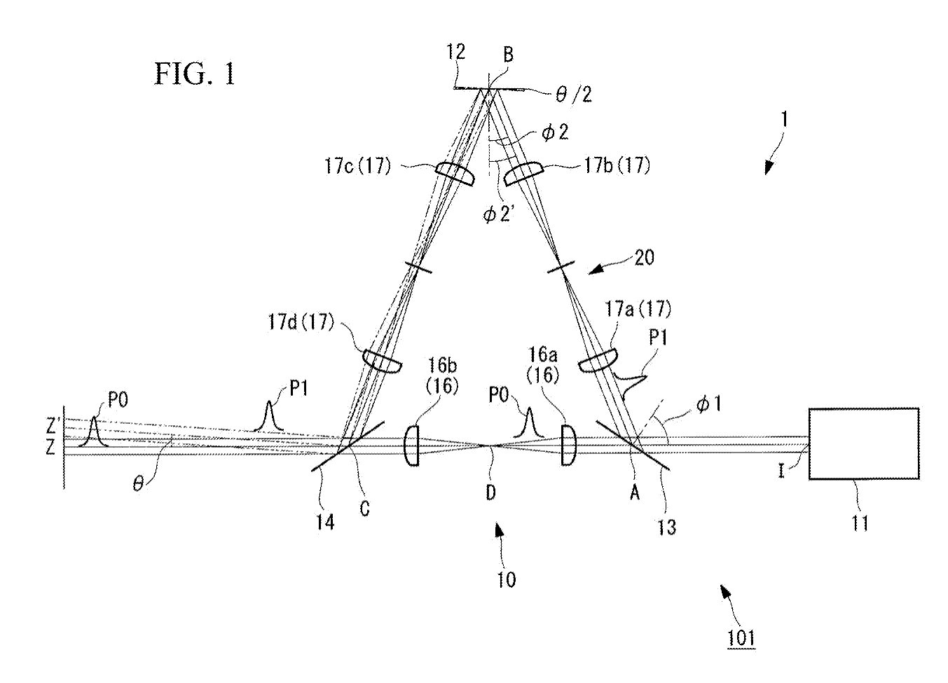

A beam splitter apparatus 1 according to a first embodiment of the present invention will now be described with reference to FIGS. 1 to 3.

As shown in FIG. 1, the beam splitter apparatus 1 according to this embodiment includes a reflection optical system (beam-angle setting section) 12, a beam splitter (branching section) 13, a beam splitter (multiplexing section) 14, and relay optical systems (pupil transfer optical systems) 16 and 17. Furthermore, the beam splitter apparatus 1 of this embodiment and a pulsed light source 11 constitute a light source apparatus 101.

In FIG. 1, the intersection points of an optical axis IZ with the reflection surfaces of the beam splitter 13 and the beam splitter 14 are referred to as point A and point C, respectively. Furthermore, the midpoint between point A and point C is referred to as point D, and the intersection point of the optical axis of a pulsed beam from the beam splitter 13 with the reflection optical system 12 is referred to as point B. Here, triangle ABC is an isosceles triangle having point B as the vertex, and side AB and side BC have the same length.

The functions of the above-mentioned components will now be described.

The pulsed light source 11 oscillates a pulsed beam with a repetition frequency R.

The beam splitter 13 is a branching section that branches the pulsed beam into two optical paths with different optical path lengths, more specifically, an optical path A-D-C (hereinafter, referred to as “an optical path 10”) and an optical path A-B-C (hereinafter, referred to as “an optical path 20”).

The reflection optical system 12 includes a mirror that totally reflects the pulsed beam from the beam splitter 13 and a swing mechanism (not shown in the figure) that swings this mirror about an axis orthogonal to the optical axis of the pulsed beam.

The reflection optical system 12 swings the mirror about an axis orthogonal to the optical axis of the pulsed beam by means of the swing mechanism, not shown in the figure, to change the angle of the optical axis of the pulsed beam branching off via the beam splitter 13.

As a result, the reflection optical system 12 functions as a stationary deflecting section that endows the pulsed beam passing along the optical path 20 branching off via the beam splitter 13 with a deflection angle of θ through tilting of the reflection surface thereof. Furthermore, the reflection optical system 12 also functions as a delaying section that delays the pulsed beam passing along the optical path 20 so that an optical path length difference L is produced between the optical path 10 and the optical path 20.

The optical path 10 and the optical path 20 include the relay optical systems 16 and 17, respectively, for relaying pupils of the pulsed beams in their respective optical paths.

The relay optical system 16 is composed of one pair of lenses 16a and 16b, and the pupil adjacent to point A is relayed to the vicinity of point C.

The relay optical system 17 is composed of two pairs of lenses 17a and 17b, and 17c and 17d, and the reflection optical system 12 is disposed between the lens 17b and the lens 17c. The lenses 17a, 17b, 17c, and 17d have the same focal length. Because of this, the pupil disposed adjacent to point A is relayed to the vicinity above the reflection optical system 12 by means of the lens 17a and the lens 17b. Furthermore, the pupil that has been relayed to the vicinity above the reflection optical system 12 is further relayed to the vicinity of point C by means of the lens 17c and the lens 17d.

The beam splitter 14 is a multiplexing section that multiplexes the pulsed beams that have passed along the optical path 10 and the optical path 20.

Although a beam splitter is used as the branching section and the multiplexing section in this embodiment, a half mirror or a dichroic mirror, for example, may be used instead. This also applies to other embodiments.

Temporal multiplexing and spatial multiplexing (spatial modulation) of a pulsed beam that has been oscillated by the pulsed light source 11 in the beam splitter apparatus 1 with the above-described structure will now be described. P Temporal multiplexing will be described first.

From point A to point Z along which a pulsed beam oscillated by the pulsed light source 11 passes, there are two optical paths: the optical path 10 having the shortest optical path length and the optical path 20 having an optical path length larger than the optical path 10 by an optical-path-length difference L. Here, the pulsed beam passing along the optical path 10 is denoted by P0, and the pulsed beam passing along the optical path 20 is denoted by P1.

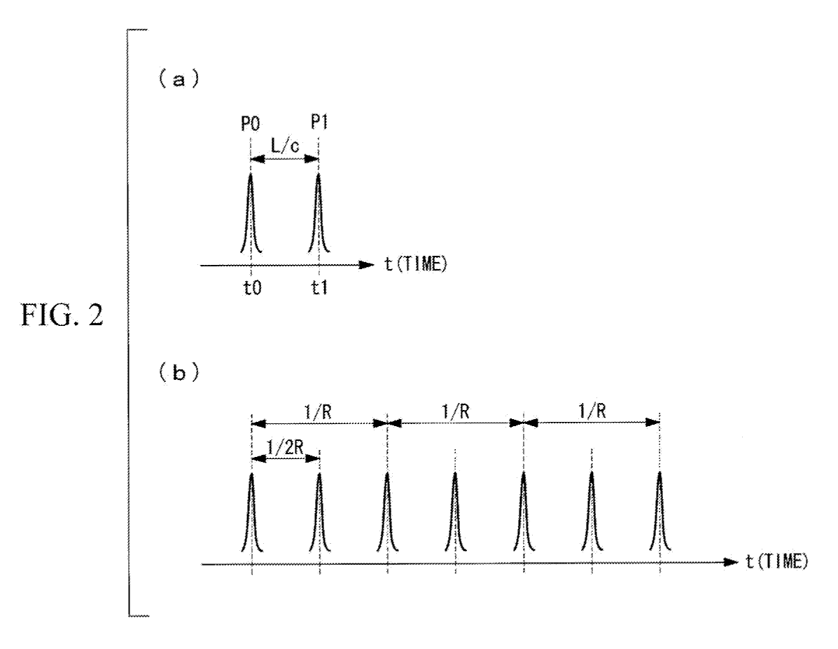

Because the optical path 20 is longer than the optical path 10 by the optical-path-length difference L, the pulsed beam P1 passing along the optical path 20 arrives at point C on the beam splitter 14 with a delay L/c compared with the pulsed beam P0 passing along the optical path 10, where c represents the velocity of light. In other words, the time t1 when the pulsed beam P1 passing along the optical path 20 arrives at point Z is expressed as t1=t0+L/c, where t0 represents the time when the pulsed beam P0 passing along the optical path 10 arrives at point Z (refer to FIG. 2(a)). Here, as shown in FIG. 2(b), when the optical-path-length difference L is selected so that L=c/2R is satisfied in relation to the repetition frequency R of the pulsed light source 11, an optical pulse train that is temporally multiplexed with a repetition frequency 2R, i.e., twice the original repetition frequency R of the pulsed light source 11, is generated.

Next, spatial multiplexing in which the pulsed beam temporally multiplexed as described above is spatially deflected will be described.

First, the following description assumes as a reference that the relative angle between the pulsed beam P0 and the pulsed beam P1 is 0 when they are multiplexed at the beam splitter 14 without spatial multiplexing.

An incident angle φ1 at the beam splitter 13 is given as follows:

φ1=(π−cos−1(d/L))/2

where side AB=side BC=L/2, side AC=d, and side AD=side DC=d/2.

At this time, an incident angle φ2 at the reflection optical system 12 is given as follows:

φ2=π/2−cos−1(d/L)

At this time, the pulsed beam P0 is temporally shifted by L/c but is not spatially shifted relative to the pulsed beam P1.

Thereafter, when the incident angle φ2 at the reflection surface of the reflection optical system 12 is converted by θ/2 to an incident angle φ2′, the pulsed beam P1 passing along the optical path 20 is deflected by θ by the reflection optical system 12. Because the pupil disposed adjacent to point B on the reflection optical system 12 is relayed to point C by the lenses 17c and 17d, the pulsed beam P1 passing along the optical path 20 is reflected at point C on the beam splitter 14 while maintaining the deflection angle θ, unlike a case where the reflection optical system 12 is not deflected, and is then propagated towards point Z′. At this time, the difference in deflection angle between side CZ and side CZ′ is θ. In other words, spatial multiplexing with deflection angles of 0 and θ can be accomplished.

Furthermore, the pupil of the pulsed beam P0 passing along the optical path 10 is relayed by the relay optical system 16.

From the description so far, a pulsed beam oscillated by the pulsed light source 11 is not only spatially multiplexed with a deflection angle interval of θ but is also temporally multiplexed being shifted by a time interval of L/c.

Because the above-described spatial multiplexing and temporal multiplexing occur at the same time in the beam splitter apparatus 1, pulsed beams produced by irradiating a space with a plurality of light beams, even if spatially overlapping one another on the detection side, can be separated on the time axis.

As described above, according to the beam splitter apparatus 1 of this embodiment, a pulsed beam oscillated from the pulsed light source 11 is branched by the beam splitter 13 into two optical paths 10 and 20 with different optical path lengths, relayed by the relay optical systems 16 and 17 disposed in the respective optical paths, and then multiplexed by the beam splitter 14. At this time, the pulsed beams P0 and P1 in the respective two optical paths 10 and 20 branching off from each other via the beam splitter 13 are endowed with a relative angle by the reflection optical system 12. By doing so, the pulsed beams P0 and P1 in the two optical paths 10 and 20, having different optical path lengths and also endowed with a relative angle, can be converged on one position.

In this case, the pupils of the pulsed beams P0 and P1 in the two optical paths 10 and 20 branching off from each other via the beam splitter 13 are relayed by the relay optical systems 16 and 17 disposed in their respective optical paths. Because of this, even when the resultant pulsed beams P0 and P1 are set to have different relative angles, the point of convergence can be prevented from shifting in the optical-axis direction. In other words, according to the beam splitter apparatus 1 of this embodiment, even with different relative angles of the pulsed beams P0 and P1, the pulsed beams P0 and P1 can be converged on the same pupil position in the optical-axis direction with a simple structure of the relay optical systems 16 and 17.

As a result, even when the relative angles of the pulsed beams P0 and P1 are changed, they can be made incident upon an optical system disposed downstream thereof under the same incidence conditions. For example, the pulsed beams P0 and P1 can be emitted to different positions on the focal plane of a microscope objective lens by converging the pulsed beams P0 and P1 endowed with a relative angle at the pupil position of the objective lens. The spacing between the radiation positions can be made different with different relative angles, and the amount of light can be prevented from fluctuating at that time.

Furthermore, because the relay optical system 16 is provided with one pair of lenses 16a and 16b, the relay optical system 17 is provided with two pairs of lenses 17a and 17b, and 17c and 17d, and the reflection optical system 12 is disposed between each of two pairs of relay lenses 17a and 17b, and 17c and 17d, the pupil is relayed by the two pairs of lenses 17a and 17b, and 17c and 17d, even when the pulsed beams P0 and P1 branching off from each other are endowed with a relative angle by the reflection optical system 12. Therefore, the point of convergence can be prevented from shifting in the optical-axis direction. In addition, by providing a plurality of pairs of such lenses and relaying pupils of the pulsed beams P0 and P1 in the two optical paths 10 and 20 with the plurality of pairs of lenses, the diameters of the lenses can be reduced.

Furthermore, a plurality of units including the beam splitter 13, the beam splitter 14, the relay optical systems 16 and 17, and the reflection optical system 12 may be provided in series, and the reflection optical system 12 may be provided between the beam splitter 13 and the beam splitter 14.

By doing so, a pulsed beam oscillated from the pulsed light source 11 can be branched into a plurality of optical paths, and the resultant pulsed beams can be endowed with a relative angle by the reflection optical system 12. As a result, pulsed beams in a plurality of optical paths having different optical path lengths and endowed with a relative angle can be converged on one position.

In addition, according to the light source apparatus 101 provided with such a beam splitter apparatus 1, a bundle of a plurality of pulsed beams, oscillated from the pulsed light source 11, having different optical path lengths, and endowed with a relative angle, can all be made to pass through the pupil position in an optical system disposed downstream thereof.

### Modification

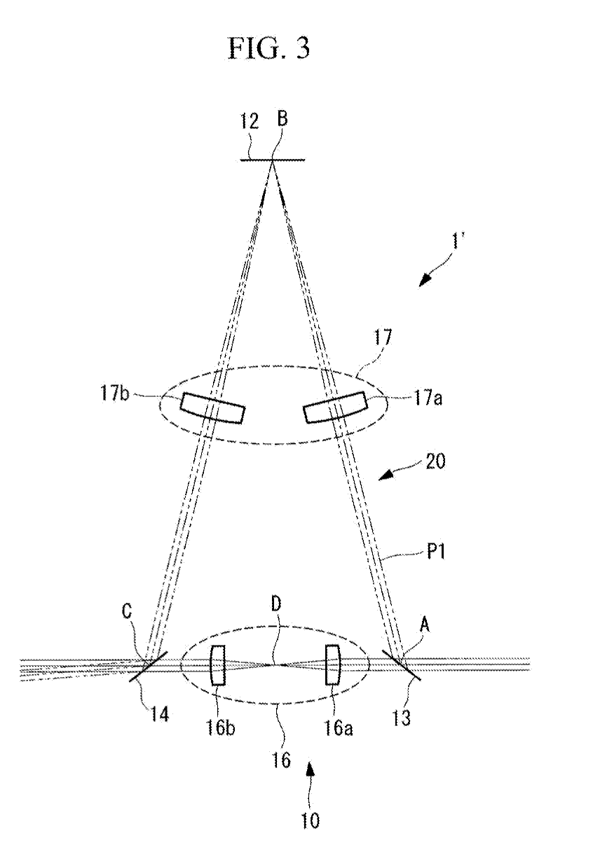

Alternatively, as a modification of this embodiment, the relay optical system 17 may be constructed with one pair of lenses 17a and 17b, and the pulsed beam P1 passing along the optical path 20 may be endowed with a deflection angle by at least one of the beam splitter 13 and the beam splitter 14, instead of the reflection optical system 12. As shown in FIG. 3, this modification will be described assuming that the pulsed beam P1 passing along the optical path 20 is endowed with a deflection angle by the beam splitter 14.

In a beam splitter apparatus 1′ according to this modification, the beam splitter 14 includes a half mirror that transmits the pulsed beam P0 passing along the optical path 10 and reflects the pulsed beam P1 passing along the optical path 20 and a swing mechanism (not shown in the figure) that swings this half mirror about an axis orthogonal to the optical axis of the pulsed beam.

The beam splitter 14 deflects and reflects the pulsed beam P1 reflected by the reflection optical system 12 by swinging the half mirror about an axis orthogonal to the optical axis of the pulsed beam P1 by the swing mechanism, not shown in the figure.

In this modification, a collimated beam that is emitted from the pulsed light source 11 and incident upon point A is branched by the beam splitter 13 into a light beam passing along the optical path 10 and a light beam passing along the optical path 20. The light beam passing along the optical path 10 is converted into a collimated beam by the relay optical system 16 but is not endowed with a deflection angle in this case. On the other hand, the light beam passing along the optical path 20 is reflected at the reflection optical system 12 disposed at point B and converted into a collimated beam by the relay optical system 17.

The beam splitter 14 multiplexes the light beam passing along the optical path 20 and the light beam passing along the optical path 10 at point C. At this time, the beam splitter 14 is endowed with a deflection angle about point C so that the light beam passing along the optical path 20 exhibits a finite angle relative to the light beam passing along the optical path 10. Because the relay optical systems 16 and 17 propagate the pupil near point A to point C, the two light beams can be made to spatially overlap each other in the vicinity of point C.

Although this modification has been described by way of an example where a deflection angle is given by the beam splitter 14, the pulsed beam P1 may be endowed with a deflection angle by either the beam splitter 13 or both the beam splitter 13 and the beam splitter 14 instead.

A pulsed light source is used in this embodiment. However, any light source is acceptable as long as it emits a pulsed beam. For example, a light source such as an LED or a laser light source that emits a laser beam may be used instead.

### REFERENCE EMBODIMENT

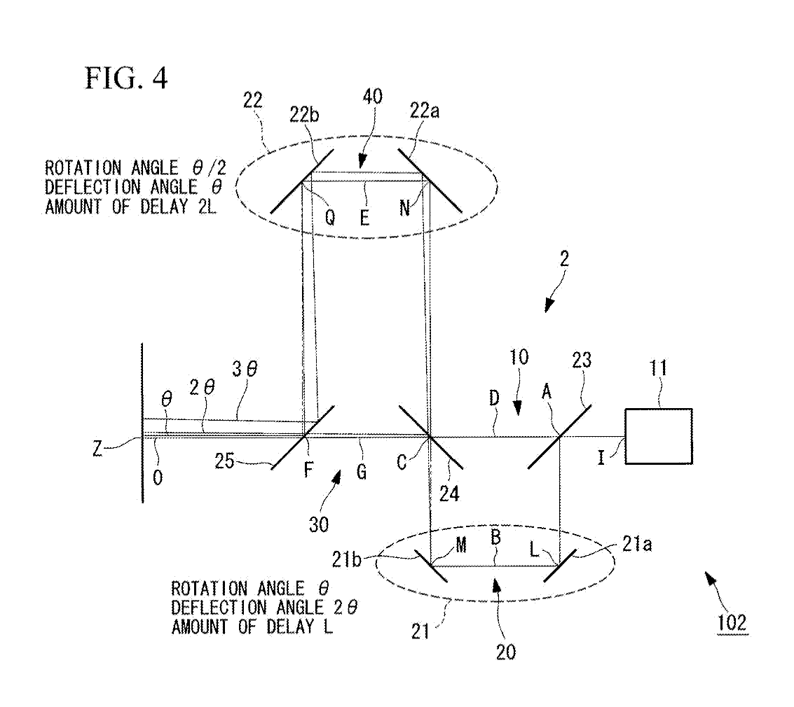

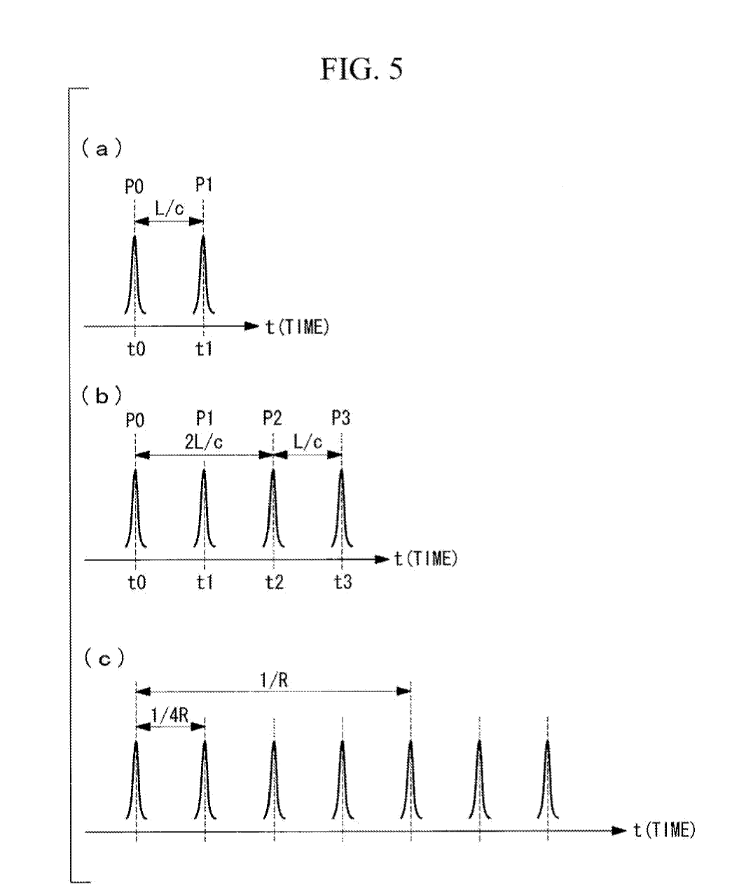

As a reference embodiment of the present invention, a beam splitter apparatus 2 will now be described with reference to FIGS. 4 and 5. In the description of this reference embodiment, commonalities with the beam splitter apparatus 1 according to the first embodiment will be omitted, and differences will be mainly described.

The beam splitter apparatus 2 according to this reference embodiment differs from the beam splitter apparatus 1 according to the first embodiment in that a beam splitter 24 that multiplexes pulsed beams in two optical paths and branches the multiplexed pulsed beams into two optical paths with different optical path lengths is provided between a beam splitter 23 and a beam splitter 25.

As shown in FIG. 4, the beam splitter apparatus 2 according to this reference embodiment includes reflection optical systems 21 and 22, the beam splitter (branching section) 23, the beam splitter (multiplexing/branching section) 24, and the beam splitter (multiplexing section) 25. Furthermore, the beam splitter apparatus 2 of this reference embodiment and the pulsed light source (laser light source) 11 constitute a light source apparatus 102.

The intersection points of the optical axis IZ of the pulsed beam oscillated from the pulsed light source 11 with the beam splitter 23, the beam splitter 24, and the beam splitter 25 are denoted by point A, point C, and point F, respectively.

Of the two optical paths branching off from each other by the beam splitter 23 between the beam splitter 23 and the beam splitter 24, the midpoint in the shorter optical path is denoted by point D, and the midpoint in the longer optical path is denoted by point B. Furthermore, of the two optical paths branching off from each other by the beam splitter 24 between the beam splitter 24 and the beam splitter 25, the midpoint in the shorter optical path is denoted by point G, and the midpoint in the longer optical path is denoted by point E.

The functions of the above-mentioned components will now be described.

The pulsed light source 11 oscillates a pulsed beam with a repetition frequency R.

The beam splitter 23 is a branching section that branches the pulsed beam into two optical paths with different optical path lengths, more specifically, an optical path A-D-C (hereinafter, referred to as “an optical path 10”) and an optical path A-B-C (hereinafter, referred to as “an optical path 20”).

The reflection optical system 21 is composed of two mirrors 21a and 21b and endows the pulsed beams passing along the two optical paths 10 and 20 branching off from each other by the beam splitter 23 with a relative angle (deflection angle) of 2θ. In addition, the reflection optical system 21 operates the two mirrors 21a and 21b to delay the pulsed beam passing along the optical path 20 so that an optical-path-length difference L is generated between the optical path 10 and the optical path 20.

The beam splitter 24 multiplexes the pulsed beams in the two optical paths 10 and 20 branching off from each other by the beam splitter 23 and also branches the multiplexed pulsed beams into two optical paths with different optical path lengths: an optical path C-G-F (hereinafter, referred to as “an optical path 30”) and an optical path C-E-F (hereinafter, referred to as “an optical path 40”).

Like the reflection optical system 21, the reflection optical system 22 is composed of two mirrors 22a and 22b and endows the pulsed beams passing along the two optical paths 30 and 40 branching off from each other by the beam splitter 24 with a relative angle (deflection angle) of θ. In addition, the reflection optical system 22 operates the two mirrors 22a and 22b to delay the pulsed beam passing along the optical path 40 so that an optical-path-length difference 2L is generated between the optical path 30 and the optical path 40.

The beam splitter 25 multiplexes the pulsed beams passing along the four optical paths 10, 20, 30, and 40.

Temporal multiplexing and spatial multiplexing (spatial modulation) of a pulsed beam that has been oscillated by the pulsed light source 11 in the beam splitter apparatus 2 with the above-described structure will be described.

Temporal multiplexing will be described first.

The pulsed light source 11 oscillates a pulsed beam with a repetition frequency R (Hz). The pulsed beam P0 oscillated at a certain point in time is branched by the beam splitter 23 disposed at point A into the two pulsed beams P0 and P1, so that the pulsed beam P0 passes along the optical path 10 and the pulsed beam P1 passes along the optical path 20. As shown in FIG. 4, because the optical path 20 has a larger optical path length than the optical path 10 by L, the pulsed beams P0 and P1 arrive at point C at different points in time. This concept is shown in FIGS. 5(a) to 5(c).

FIG. 5(a) depicts a time delay produced by the reflection optical system 21, FIG. 5(b) depicts a time delay produced by the reflection optical system 22, and FIG. 5(c) depicts an optical pulse train.

In FIGS. 5(a) to 5(c), the time when the pulsed beam P0 arrives at point C is denoted by an arrival time t0. Because the difference in optical path length between the optical path 10 and the optical path 20 is L, the pulsed beam P1 arrives at point C at a time t1, with a delay of L/c from the time t0, where c represents the velocity of light.

Both the pulsed beams P0 and P1 are multiplexed by the beam splitter 24 disposed at point C, and the beam splitter 24 also branches the pulsed beams P0 and P1. Because of this, each of the pulsed beams P0 and P1 propagates along the two optical paths serving as the optical path 30 and the optical path 40. As shown in FIG. 4, because the optical path 40 has a larger optical path length than the optical path 30 by 2L, the pulsed beams P0 and P1 arrive at point F with a time difference of 2L/c between a case where they pass along the optical path 40 and a case where they pass along the optical path 30. Here, the pulsed beams P0 and P1 passing along the optical path 40 are renamed pulsed beams P2 and P3, respectively.

Consequently, there are four paths from point A to point Z, and the pulsed beams P0 to P3 arrive in the vicinity of point Z via any one of the following optical paths:

Optical path 10 (P0): A-D-C-G-F-Z (shortest optical path length)

Optical path 20 (P1): A-B-C-G-F-Z

Optical path 30 (P2): A-D-C-E-F-Z

Optical path 40 (P3): A-B-C-E-F-Z

Because the beam splitter 25, constituting a multiplexing section, is disposed at point F, the four pulsed beams P0 to P3 are multiplexed with their optical axes oriented towards point Z. Therefore, as shown in FIG. 5(b), temporal multiplexing in the form of pulsed beams at regular intervals on the time axis is accomplished at the time of arrival at point Z. Here, as shown in FIG. 5(c), when the optical-path-length difference L is selected so that L=c/4R is satisfied in relation to the repetition frequency R of the pulsed light source 11, an optical pulse train that is temporally multiplexed with a repetition frequency of 4R is generated.

Next, spatial multiplexing in which the pulsed beam temporally multiplexed as described above is spatially deflected will be described.

In this reference embodiment, the reflection surfaces of the beam splitters 23, 24, and 25 and the two mirrors 21a and 21b of the reflection optical system 21 are disposed so as to have an angle of 45° relative to the optical axis IZ. Quadrangle ALMC is a rectangle, where L and M represent the centers of the mirrors 21a and 21b, respectively, of the reflection optical system 21. Therefore, when the pulsed beam P1 passing along the optical path 20 is multiplexed with the pulsed beam P0 by the beam splitter 24, the deflection angle between the pulsed beam P0 and the pulsed beam P1 is 0 relative to the completely coaxial state serving as a reference. On the other hand, when at least one mirror of the reflection optical system 21 is rotated by a rotation angle of θ relative to the reference state, as shown in FIG. 4, the pulsed beam P1 arrives at point C with a deflection angle of 2θ. FIG. 4 shows a case where only 21b is rotated.

Therefore, when the pulsed beams P0 and P1 are multiplexed at the beam splitter 24, the two pulsed beams exhibit a deflection angle of 2θ immediately after they have entered the optical path 30 and the optical path 40. In the same manner, when at least one mirror of the reflection optical system 22 is rotated by a rotation angle of θ/2, the pulsed beams P2 and P3 having a deflection angle of θ relative to the pulsed beams P0 and P1 are multiplexed at the beam splitter 25. FIG. 4 shows a case where only 22b is rotated.

The pulsed beam P2 is deflected by the reflection optical system 22 so as to have a deflection angle of θ after the pulsed beam P0 has been branched at point C. On the other hand, the pulsed beam P3 is produced as a result of the pulsed beam P1 being endowed with a deflection angle of θ at the reflection optical system 22. Because the pulsed beam P3 has been endowed with a deflection angle of 2θ at the reflection optical system 21, it has a total deflection angle of 3θ. Consequently, as shown in FIG. 4, the pulsed beams P0, P1, P2, and P3 propagate in the directions with deflection angles of 0, 2θ, θ, and 3θ relative to the optical axis IZ, thus accomplishing spatial multiplexing.

In this reference embodiment, the deflection angle is 2θ when the amount of delay (the difference in optical path length) is L, and the deflection angle is θ when the amount of delay is 2L. Therefore, when the amounts of delay of the pulsed beams P0, P1, P2, and P3 are 0, L, 2L, and 3L, the respective deflection angles are 0, 2θ, θ, and 3θ.

Because the above-described spatial multiplexing and temporal multiplexing occur at the same time in the beam splitter apparatus 2, the pulsed beam emitted from the pulsed light source 11 exhibits temporal multiplexing with a time interval of L/c and spatial multiplexing with a deflection angle interval of θ.

As described above, according to the beam splitter apparatus 2 of this reference embodiment, the beam splitter 24 that branches and multiplexes pulsed beams is provided so that an input pulsed beam can be branched into a plurality of optical paths by the beam splitter 23 and the beam splitter 24 and so that the resultant pulsed beams can be endowed with a relative angle by the reflection optical systems 21 and 22. By doing so, pulsed beams in a plurality of optical paths, having different optical path lengths and also endowed with a relative angle, can be produced.

In addition, because one pulsed beam can be multiplexed to four in this reference embodiment, the signal acquisition level per unit time is increased. This helps achieve fast image generation processing when it is applied to, for example, a microscope.

Although this reference embodiment has been described by way of an example where one beam splitter 24 for branching and multiplexing pulsed beams is provided, two or more beam splitters may be provided. By doing so, a pulsed beam from the pulsed light source 11 can be branched into a larger number of beams, thereby further increasing the speed of image generation processing.

### Second Embodiment

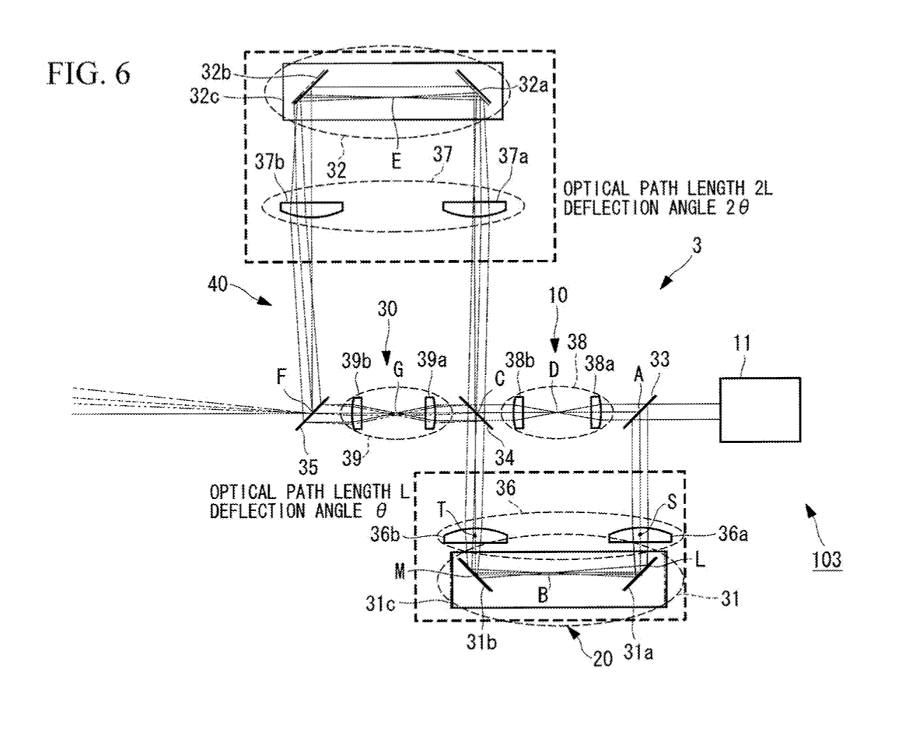

A beam splitter apparatus 3 according to a second embodiment of the present invention will now be described with reference to FIGS. 6 to 8. In the description of this embodiment, commonalities with the above-described embodiment will be omitted, and differences will be mainly described.

The beam splitter apparatus 3 according to this embodiment differs from the beam splitter apparatus 2 according to the reference embodiment in that relay optical systems (pupil transfer optical systems) 36, 37, 38, and 39 serving as means for propagating the pupil position is provided.

As shown in FIG. 6, the beam splitter apparatus 3 according to this embodiment includes reflection optical systems 31 and 32; a beam splitter (branching section) 33; a beam splitter (multiplexing/branching section) 34; a beam splitter (multiplexing section) 35; and the relay optical systems 36, 37, 38, and 39 serving as means for propagating the pupil position. Furthermore, the beam splitter apparatus 3 of this embodiment and the pulsed light source 11 constitute a light source apparatus 103.

The relay optical systems 36, 37, 38, and 39 each include one pair of lenses and are disposed one each in the branching optical paths. The relay optical systems 36, 37, 38, and 39 relay the pupils of pulsed beams in their respective optical paths.

More specifically, the relay optical system 36, for example, is composed of one pair of lenses 36a and 36b to relay the pupil of the pulsed beam passing along the optical path 20 branching off via the beam splitter 33. In the same manner, the relay optical systems 37, 38, and 39 include one pair of lenses 37a and 37b, one pair of lenses 38a and 38b, and one pair of lenses 39a and 39b, respectively, to relay the pupils of the pulsed beams passing along the optical paths branching off via the beam splitter 33 or the beam splitter 34.

The reflection optical system 31 includes a mirror (first mirror) 31a that reflects the pulsed beam branching off via the beam splitter 33; a mirror (second mirror) 31b that reflects the pulsed beam reflected at the mirror 31a towards the beam splitter 34; and a stage (rectilinear translation mechanism) 31c that rectilinearly translates these mirrors 31a and 31b together in the optical-axis direction between these mirrors.

The reflection optical system 31 rectilinearly translates the mirrors 31a and 31b together in the optical-axis direction between these mirrors by means of the stage 31c to endow the pulsed beam branching off via the beam splitter 33 with a difference in optical path length, as well as a deflection angle.

In the same manner, the reflection optical system 32 includes a mirror (first mirror) 32a that reflects the pulsed beam branching off via the beam splitter 34; a mirror (second mirror) 32b that reflects the pulsed beam reflected at the mirror 32a towards the beam splitter 35; and a stage (rectilinear translation mechanism) 32c that rectilinearly translates these mirrors 32a and 32b together in the optical-axis direction between these mirrors.

The reflection optical system 32 rectilinearly translates the mirrors 32a and 32b together in the optical-axis direction between these mirrors by means of the stage 32c to endow the pulsed beam branching off via the beam splitter 34 with a difference in optical path length, as well as a deflection angle.

Temporal multiplexing and spatial multiplexing (spatial modulation) of a pulsed beam that has been oscillated by the pulsed light source 11 in the beam splitter apparatus 3 with the above-described structure will be described.

Because temporal multiplexing can be accomplished by following an adjustment procedure similar to that described in the foregoing reference embodiment, a description thereof will be omitted. Thus, spatial multiplexing will be described below.

The relay optical systems 36, 37, 38, and 39 are each composed of a lens pair including two lenses having the same focal length to form an image of the pupil disposed adjacent to point A of the beam splitter 33 in the vicinity of point C on the beam splitter 34. Furthermore, they form an image of the pupil disposed adjacent to point C on the beam splitter 34 in the vicinity of point F on the beam splitter 35. Here, assuming that the optical path A-D-C (hereinafter, referred to as “the optical path 10”) and the optical path C-G-F (hereinafter, referred to as “the optical path 30”) have the same optical path length L1, the focal length f1 of the lenses of the lens pairs used in the relay optical systems 38 and 39 is selected so as to satisfy f1=L1/4.

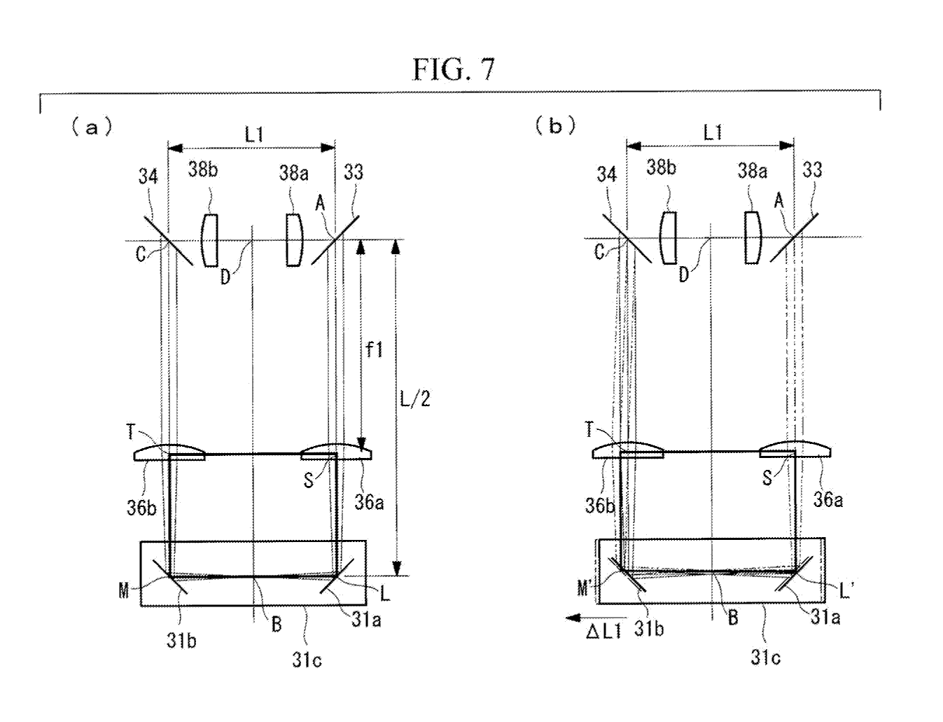

FIG. 7(a) depicts an arrangement where the pulsed beam is not deflected.

With reference to FIG. 7(a), the relationship between the amount of delay L in the optical path A-B-C (hereinafter, referred to as “the optical path 20”) and the focal length f1 of the lenses in the relay optical system 36 will be described. It is assumed that the points of incidence of the principal rays upon the two lenses 36a and 36b provided in the relay optical system 36 are denoted by S and T and that the points of reflection of the principal rays at the two mirrors 31a and 31b provided in the reflection optical system 31 are respectively denoted by L and M. The quadrangle ALMC formed by connecting these four points is a rectangle with all angles of 90° when no deflection is performed. In this case, because side LM and side AC have the same length, the given amount of delay L is equal to the sum of side AL and side MC and is accordingly equal to 2AL. More specifically, the focal length f1 of the lenses is f1=(L1+L)/4, and the mirrors and lenses are arranged so that the two given optical paths satisfy AS=SL+LB=BM+MT=TC=f1.

The pulsed beam reflected at the beam splitter 33 passes via the lens 36a, the mirror 31a, the mirror 31b, and the lens 36b in that order and is then multiplexed by the beam splitter 34 with the pulsed beam passing through the relay optical system 38.

FIG. 7(b) depicts an arrangement where a pulsed beam is deflected.

In the reflection optical system 31, the mirror 31a and the mirror 31b face each other such that they are tilted with an angle of 45° relative to the optical axis AZ and are disposed on the stage 31c that can be moved in a direction parallel to the optical axis AZ. As shown in FIG. 7(b), when the stage 31c is moved in the direction indicated by the arrow, the line segment L′M′ formed by connecting the points of reflection of principal rays at the mirrors 31a and 31b not only moves towards the lenses relative to the line segment LM assumed when no deflection is performed but also shifts in a direction indicated by the arrow. As a result, the principal ray of the pulsed beam reflected at point M′ of the mirror 31b shifts leftwards compared with a case where no deflection is performed and, after having passed through the lens 36b, is converted into a collimated beam deflected relative to the optical axis MC of the lens. Because the displacement of the optical axis is twice the displacement of the stage (i.e., 2ΔL1), this deflection angle θ satisfies the relation tan θ=2ΔL1/f1, where ΔL1 represents the displacement of the stage 31c.

Likewise, a relationship between the amount of delay 2L and the focal length f2 of the lenses in the relay optical system 37 also holds in the optical path 40. More specifically, the focal length f2 of the lenses used in the relay optical system 37 is obtained from f2=(L1+2L)/4, and the displacement ΔL2 of the stage 32c is set so as to satisfy tan 2θ=2ΔL2/f2.

From the description so far, adjustment is performed so that the deflection angle in the optical path 20 is θ and the deflection angle in the optical path 40 is 2θ.

Because the above-described spatial multiplexing and temporal multiplexing occur at the same time in the beam splitter apparatus 3, the pulsed beam emitted from the pulsed light source 11 exhibits temporal multiplexing with a time interval of L/c and spatial multiplexing with a deflection angle interval of θ.

The beam splitter apparatus 3 according to this embodiment differs from the beam splitter apparatus 2 according to the reference embodiment in that relay optical systems are used. When relay optical systems are used as in this embodiment, pulsed beams having four deflection angles can be made to spatially overlap one another in the vicinity of the branching section or the multiplexing section by the effect of propagating the pupil positions. As a result, the size of the optical element used for branching and multiplexing can be reduced.

Furthermore, a figure formed by optical paths in which only mirrors are disposed exhibits a trapezoidal shape, which is a deformation of a rectangle. When a deflection angle is changed, the shape of the trapezoid also changes, causing the optical paths to differ from one another. As a result, because the time difference when the pulsed beams P0 and P1 are multiplexed differs depending on the deflection angle, changing the interval for spatial multiplexing causes the interval for temporal multiplexing also to change. In contrast, because formation of a pupil image is performed by the lenses of the pupil propagating section in this embodiment, the pupil and the pupil image are optically conjugate. For this reason, the optical path 20 does not change even when the deflection angle is changed. Therefore, the interval for spatial multiplexing alone can be changed by modulating only the deflection angle while keeping the time intervals of a pulse train formed by the pulsed beams P0, P1, P2, and P3 fixed.

Although four relay optical systems are used in this embodiment, one relay optical system may be used. In that case, the one relay optical system is most effectively disposed at the position of the relay optical system 37. The reason for this will be described below. Normally, a pulsed beam does not propagate in the form of a completely collimated beam but propagates with a slight diverging angle. Therefore, when beams passing along paths with different optical path lengths are multiplexed, as in this embodiment, a wide diversity of beam diameter sizes will result due to divergence of beams passing along the shortest to the longest optical paths. To prevent this, it is a good idea to place the one relay optical system in the longest optical path to correct the spread due to divergence. Therefore, it is most effective that the relay optical system is disposed at the position of the relay optical system 37. Furthermore, it is desirable that a relay optical system be placed in all optical paths in order to make the beam diameters strictly uniform.

### Modification

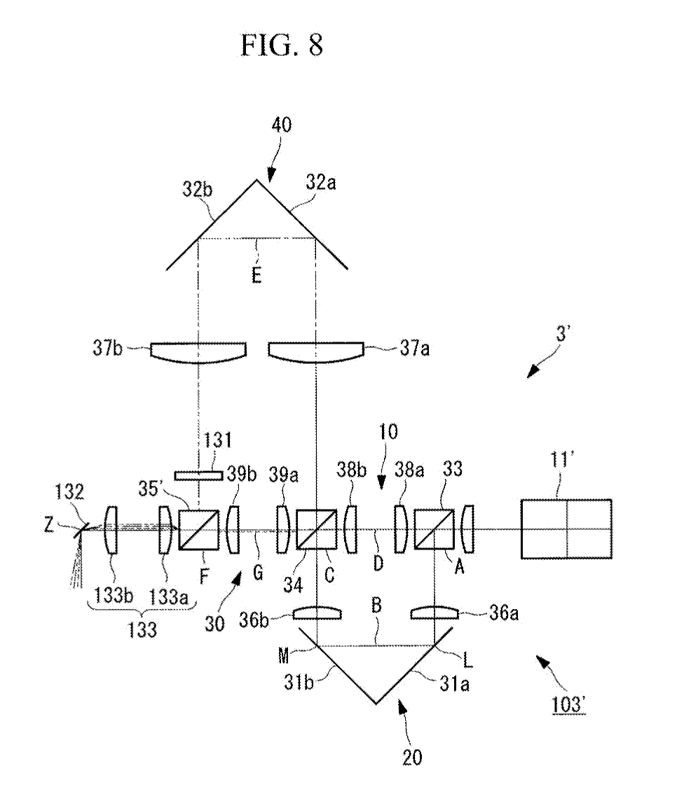

FIG. 8 shows a beam splitter apparatus 3′ according to a modification of the second embodiment.

In comparison with the beam splitter apparatus 3 according to the second embodiment, a polarizing beam splitter 35′ is employed instead of the beam splitter 35, a λ/2 plate 131 is additionally provided as a polarization modulator, and a movable mirror 132 is additionally provided as a variable deflecting section. Furthermore, a relay optical system 133 serving as a pupil transfer section is additionally provided immediately downstream of the polarizing beam splitter 35′.

The procedures for temporal multiplexing and spatial multiplexing are the same as in the second embodiment. In the second embodiment, while most of the pulsed beams multiplexed at point F on the beam splitter 35 travel towards point Z, some of the same pulsed beams propagate in a direction orthogonal to the optical axis AZ (not shown in the figure). In short, some of the pulsed beams do not proceed in the intended direction. In this modification, the loss of the pulsed beam can be minimized by adjusting the polarization.

A pulsed light source 11′ oscillates a p-polarized pulsed beam. Thereafter, the p-polarized pulsed beam travels to just before the polarizing beam splitter 35′ in the same manner as in the second embodiment. Here, the pulsed beam passing along the optical path 40 is modulated from p-polarized light to s-polarized light by the λ/2 plate 131. Consequently, the pulsed beams P0 and P1 are p-polarized light, whereas P2 and P3 are s-polarized light. For this reason, all s-polarized pulsed beams are reflected at the polarizing beam splitter 35′, whereas all p-polarized pulsed beams pass through the polarizing beam splitter 35′, thus causing all pulsed beams to be guided in the Z direction.

Furthermore, the pulsed beams multiplexed at the polarizing beam splitter 35′ are relayed to the reflection surface of the movable mirror 132 by the relay optical system 133. The movable mirror 132 has a rotation axis orthogonal to the drawing, and when it is continuously deflected from angles 0 to θ in the drawing with this movable mirror, scanning can be performed within an angular range from 0 to 4θ in the drawing.

As described above, according to the beam splitter apparatus 3′ of this modification, the polarization states of the optical paths 30 and 40 can be made orthogonal to each other by the λ/2 plate 131, and all pulsed beams passing along the two optical paths 30 and 40 are multiplexed by the polarizing beam splitter 35′ because the multiplexing section is formed of the polarizing beam splitter 35′, thereby enabling the loss of the intensity of these pulsed beams to be suppressed, which increases the utilization efficiency of the input pulsed beams.

### Third Embodiment

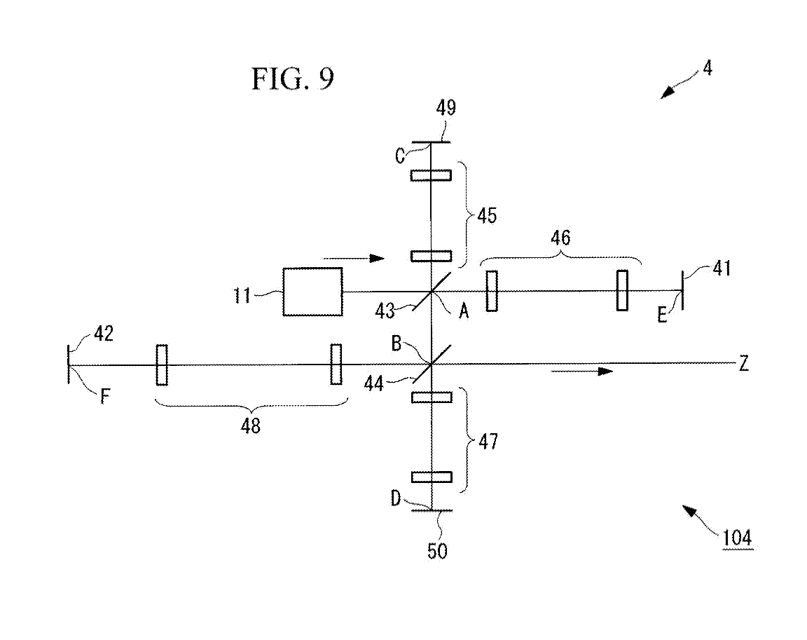

A beam splitter apparatus 4 according to a third embodiment of the present invention will now be described with reference to FIG. 9. In the description of this embodiment, commonalities with the above-described embodiments will be omitted, and differences will be mainly described.

In each of the above-described embodiments, pulsed beams pass along a plurality of optical paths of combined rectangular optical paths and straight optical paths. In this embodiment, on the other hand, a Michelson interferential optical path is used for the optical paths of pulsed beams.

As shown in FIG. 9, the beam splitter apparatus 4 according to this embodiment includes reflection optical systems (beam-angle setting sections) 41 and 42 composed of one mirror; beam splitters (multiplexing/branching sections) 43 and 44; relay optical systems (pupil transfer optical system) 45, 46, 47, and 48; and stationary mirrors 49 and 50. Furthermore, the beam splitter apparatus 4 of this embodiment and the pulsed light source 11 constitute a light source apparatus 104.

In FIG. 9, there are four optical paths as listed below:

Optical path 10: A-C-A-B-D-B-Z

Optical path 20: A-E-A-B-D-B-Z

Optical path 30: A-C-A-B-F-B-Z

Optical path 40: A-E-A-B-F-B-Z

The optical path A-E-A has a larger optical path length than the optical path A-C-A by L, and similarly, the optical path B-F-B has a larger optical path length than the optical path B-D-B by 2L. Therefore, the pulsed beams passing along the optical paths 10 to 40 up to point Z are temporally multiplexed with a time difference of L/c, as in each of the above-described embodiments. Furthermore, the relay optical systems 45, 46, 47, and 48 function to establish an optically conjugate relationship between points A and C, points A and E, points B and D, and points B and F, respectively, so that the pupils are propagated.

In this embodiment, the reflection optical systems 41 and 42 work as stationary deflecting sections. The tilt angle is changed to θ/2 by the reflection optical system 41 and to θ by the reflection optical system 42 to allow the reflection optical systems to endow pulsed beams with deflection angles of θ and 2θ, respectively. By doing so, four pulsed beams arriving at point E are spatially multiplexed with deflection angles of 0, θ, 2θ, and 3θ.

According to the beam splitter apparatus 4 of this embodiment, because optical elements are arranged along a straight line in each of the four optical paths, optical adjustment can be accomplished easily.

### Fourth Embodiment

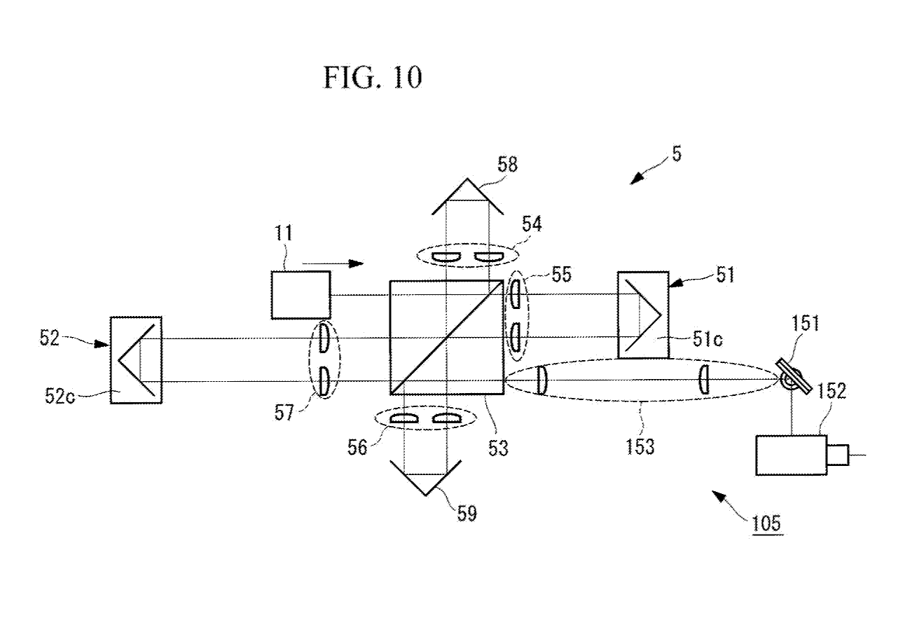

A beam splitter apparatus 5 according to a fourth embodiment of the present invention will now be described with reference to FIG. 10. In the description of this embodiment, commonalities with the above-described embodiments will be omitted, and differences will be mainly described.

As shown in FIG. 10, the beam splitter apparatus 5 according to this embodiment includes reflection optical systems (beam-angle setting sections) 51 and 52 composed of two mirrors; a beam splitter (multiplexing/branching section) 53; relay optical systems (pupil transfer optical systems) 54, 55, 56, 57, and 153; stages 51c and 52c; a pair of stationary mirrors 58 and 59; and movable mirrors 151 and 152. Furthermore, the beam splitter apparatus 5 of this embodiment and the pulsed light source 11 constitute a light source apparatus 105.

Differences from the above-described third embodiment will be described mainly.

In the beam splitter apparatus 5 according to this embodiment, the same beam splitter 53 is used as all means for performing branching and multiplexing. In addition, two-dimensional scanning can be accomplished by using the movable mirrors 151 and 152 in respective light-guide directions of multiplexed pulsed beams.

With the above-described structure, because all branching and multiplexing operations are accomplished with just one beam splitter 53, the number of components can be reduced.

### Modification

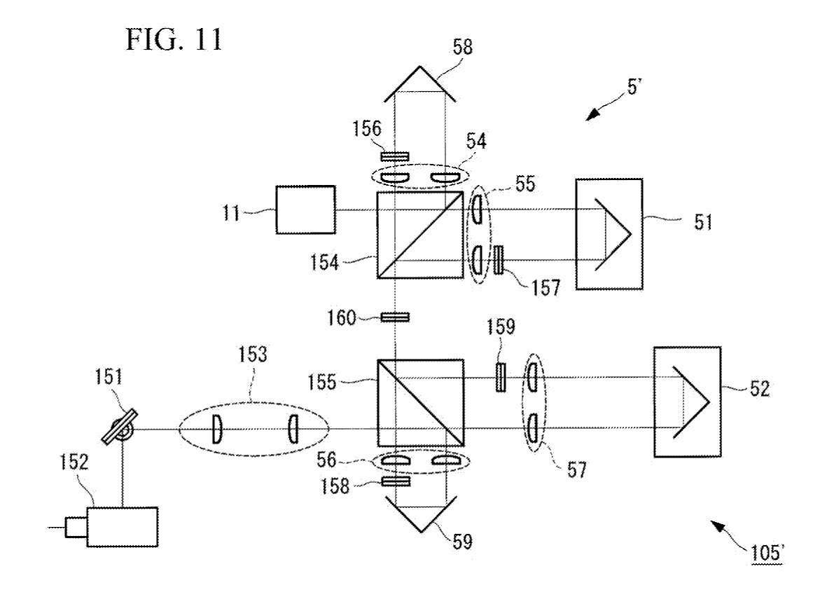

Alternatively, like the modification of the second embodiment, the loss of pulsed beams may be minimized through polarization adjustment. In this case, polarizing beam splitters 154 and 155 are arranged as shown in a beam splitter apparatus 5′ of FIG. 11. In addition, λ/2 plates 156, 157, 158, and 159 are disposed in four respective optical paths so as to achieve a polarization of 90° after the end of the branching operation, and furthermore, a λ/2 plate 160 that achieves a polarization of 45° is disposed in the optical path between the polarizing beam splitters 154 and 155.

### Fifth Embodiment

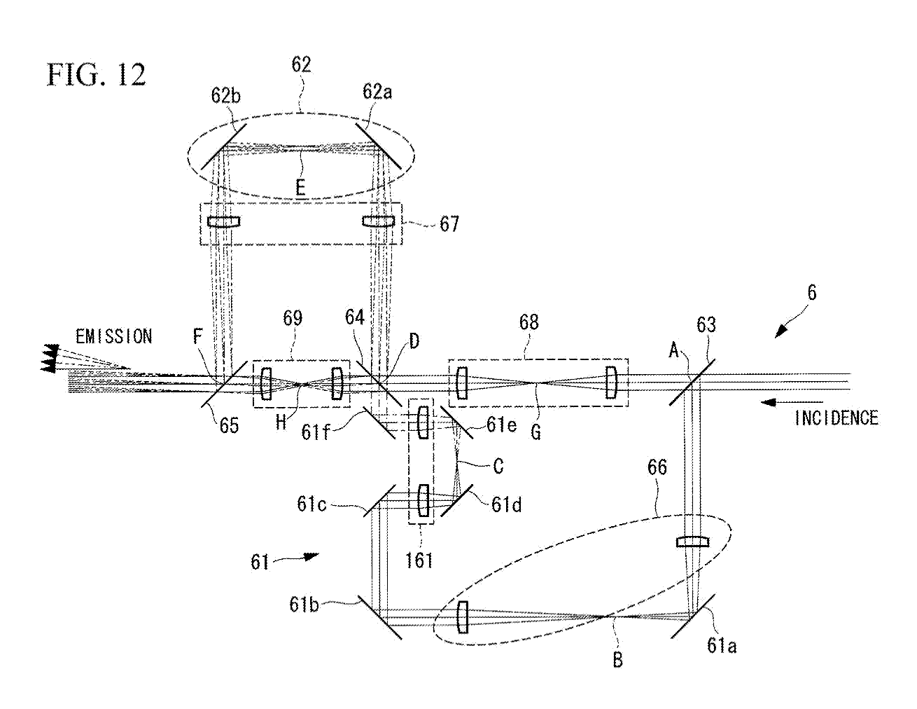

A beam splitter apparatus 6 according to a fifth embodiment of the present invention will now be described with reference to FIG. 12. In the description of this embodiment, commonalities with the above-described embodiments will be omitted, and differences will be mainly described.

The beam splitter apparatus 6 according to this embodiment includes reflection optical systems 61 and 62; beam splitters 63, 64, and 65; and relay optical systems 66 to 69 and 161.

The reflection optical system 61 denotes reflection optical systems (mirrors) 61a to 61f disposed in the optical path A-B-C-D produced by the beam splitter 63, and the reflection optical system 62 denotes reflection optical systems (mirrors) 62a and 62b disposed in the optical path D-E-F produced by the beam splitter 64.

The relay optical system 68 relays the pupil adjacent to point A along the optical path A-G-D produced by the first branching operation. The relay optical systems 66 and 161 relay the pupil adjacent to point A along the optical path A-B-C-D.

Likewise, the relay optical systems 69 and 67 relay the pupil adjacent to point D along the optical path D-H-F and the optical path D-E-F, respectively.

In this embodiment, two sets of the relay optical systems 66 and 161 are provided in the longer path A-B-C-D of the two delay paths. The reason for this is described below. Assuming that a deflection angle is θ and the focal length of a relay optical system is f, the aperture radius of the relay optical system required to efficiently propagate a collimated beam endowed with the deflection angle needs to be larger than the sum of f tan θ and the beam radius. In other words, when a pupil is to be propagated along a delay path by one set of relay optical systems, as in each of the above-described embodiments, a larger focal length inevitably requires a larger aperture of the relay optical system. For this reason, an optical system having a large aperture needs to be prepared.

In this embodiment, the pupil adjacent to point A is relayed by the relay optical system 66 having a very large focal length and the relay optical system 161 having a small focal length. A deflection angle is produced by moving the reflection optical systems 61d and 61e in the optical axis direction between these reflection optical systems. Because a small focal length is selected for the relay optical system 161, the aperture sizes of the relay optical systems 66 and 161 can be prevented from becoming large.

### Sixth Embodiment

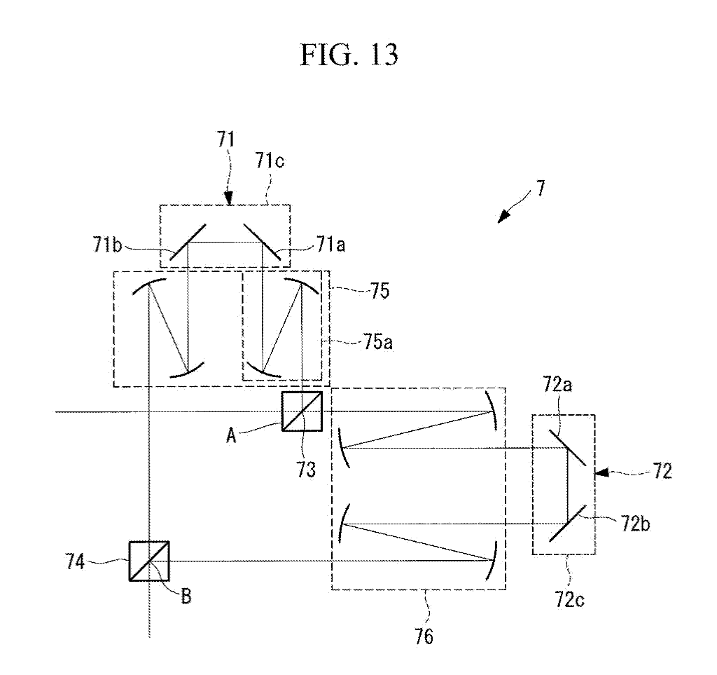

A beam splitter apparatus 7 according to a sixth embodiment of the present invention will now be described with reference to FIG. 13. In the description of this embodiment, commonalities with the above-described embodiments will be omitted, and differences will be mainly described.

The beam splitter apparatus 7 according to this embodiment includes reflection optical systems 71 and 72; beam splitters 73 and 74; and relay optical systems 75 and 76 composed of reflection elements. The relay optical systems 75 and 76 shown here are composed of two reflection optical systems 75a formed of two non-flat reflection surfaces to relay the pupils of pulsed beams in their respective optical paths.

The reflection optical system 71 rectilinearly translates the mirrors 71a and 71b together in the optical-axis direction between these mirrors by means of the stage 71c to endow the pulsed beam branching off via the beam splitter 73 with a difference in optical path length, as well as a deflection angle.

The reflection optical system 72 rectilinearly translates the mirrors 72a and 72b together in the optical-axis direction between these mirrors by means of the stage 72c to endow the pulsed beam branching off via the beam splitter 73 with a difference in optical path length, as well as a deflection angle.

The relay optical systems 75 and 76 need not be transmissive (refractive), as shown here, but may be reflective. Furthermore, although two optical systems with positive refractive power are provided as a structure for relaying along the path from point A to point B, positive and negative power may be combined.

### Seventh Embodiment

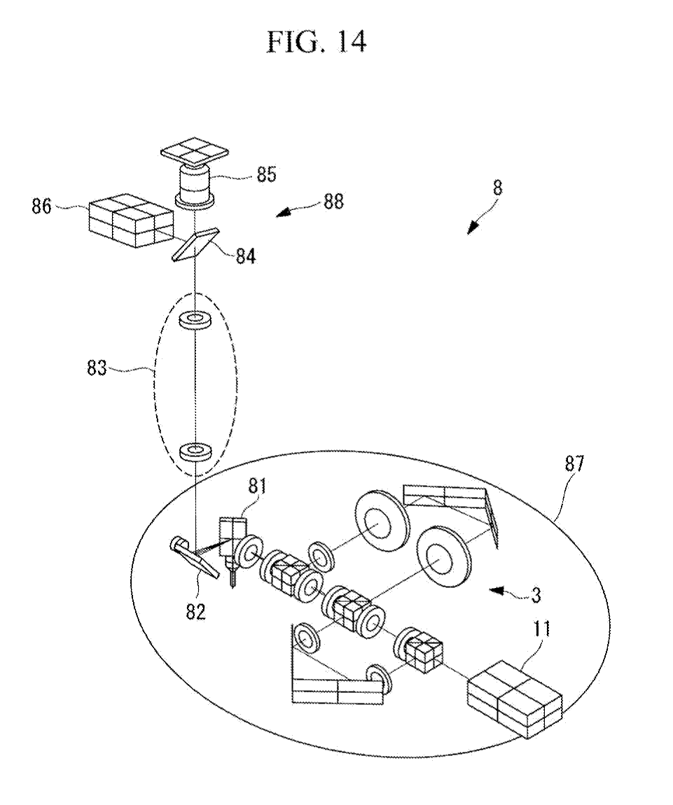

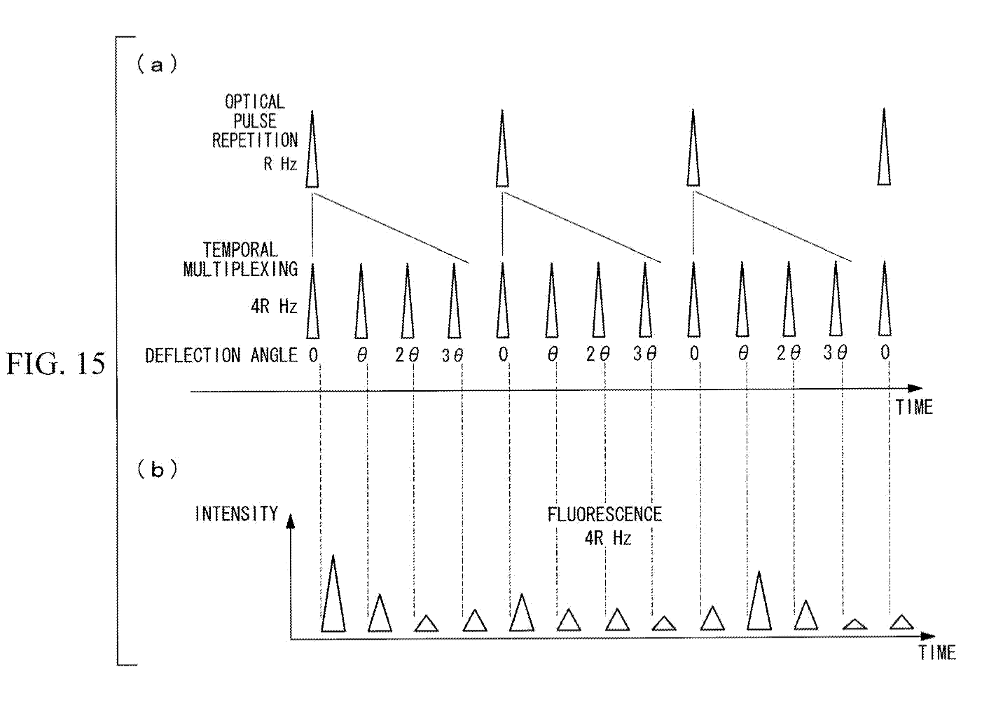

As a seventh embodiment according to the present invention, an example where the above-described beam splitter apparatus is applied to a scanning microscope will be described with reference to FIGS. 14 and 15.

As shown in FIG. 14, a scanning microscope 8 according to this embodiment includes the beam splitter apparatus 3 with the same structure as in the second embodiment; the pulsed light source 11; movable mirrors 81 and 82; a relay lens 83; a dichroic mirror 84; an objective lens 85; and a detector 86. Although not shown in the figure, the scanning microscope 8 further includes a processing section for synchronizing detection timing by the detector 86 with the pulsed light source 11; a restoring section; and a display section.

The beam splitter apparatus 3, the pulsed light source 11, and the movable mirrors 81 and 82 constitute a scanning optical system (scanning section) 87 that scans a subject with a plurality of pulsed beams from the beam splitter apparatus 3.

Furthermore, the relay lens 83, the dichroic mirror 84, and the objective lens 85 constitute an observation optical system 88 that irradiates the subject with pulsed beams scanned by the scanning optical system 87 and collects light from the subject.

The detector 86 is a detecting section that detects light collected by the observation optical system 88.

As described in the second embodiment, pulsed beams are endowed with respective deflection angles of 0, θ, 2θ, and 3θ by the reflection optical systems 31 and 32 in the beam splitter apparatus 3. In this manner, a deflection angle is assigned to each pulsed beam by the beam-angle setting section and those pulsed beams are multiplexed to form an optical pulse train (spatial multiplexing).

When one pulsed beam is converted into a plurality of (four) spatially multiplexed pulsed beams, a plurality of sites on the subject can be irradiated with those pulsed beams, and therefore, a scanning speed four times as high as when the subject is scanned with a single pulsed beam can be accomplished.

Furthermore, while a pulsed beam emitted from the pulsed light source 11 at a repetition frequency R Hz is branched by the branching section, the resultant pulsed beams pass along optical paths with different optical path lengths. As a result, the pulsed beams form an optical pulse train at regular temporal intervals (temporal multiplexing). The optical path lengths are made to differ from one another at the branches, for example, in the beam splitter apparatus 3, so that the formed overall optical pulse train has a frequency of 4R, as shown in FIG. 15(a).

When this optical pulse train is radiated onto sites of the subject, fluorescence is produced for each pulsed beam by multiphoton excitation effect. Because this fluorescence is produced immediately after each pulsed beam of the optical pulse train is radiated, fluorescent signal light with a period of frequency 4R occurs as shown in FIG. 15(b).

This fluorescent signal light (one-dimensional time information) with a frequency of 4R is collected by the observation optical system 88 as fluorescent signal light from the subject and is detected by the detector 86. Thereafter, the detected fluorescent signal light is synchronized with the optical pulse train by the processing section (not shown in the figure), is associated as fluorescent signals for respective sites of the subject, and is reconstructed into two-dimensional information by the restoring section (not shown in the figure). Subsequently, the subject can be imaged when this two-dimensional information is displayed on the display section (not shown in the figure). Although two-dimensional information is obtained in this embodiment because signal light based on two-dimensional scanning is reconstructed, three-dimensional information can be obtained by performing three-dimensional scanning.

However, if the interior of a subject is to be examined when the subject is a scatterer, signal light produced from irradiated sites spreads widely, leading to a wide distribution of light on the detector 86. Therefore, the resolving power will decrease because signal beams from various sites are mixed if temporal multiplexing is not performed.

However, according to the scanning microscope 8 of this embodiment, because the optical path lengths are made to differ from one another (temporal multiplexing) for respective pulsed beams in the beam splitter apparatus 3, as shown in FIG. 15(b), fluorescent signal light beams produced from sites arrive at the detector at different frequencies corresponding to the respective irradiated pulsed beams.

Because fluorescent signal light beams from sites correspond to respective pulsed beams, they can be separated easily in the time domain through synchronization by the processing section, and therefore, the correspondence relationship between pulsed beams radiated onto sites and the resultant fluorescent signal light beams is elucidated. Because it is possible to identify which site of the subject is irradiated with a pulsed beam for a particular fluorescent signal light beam originating from that pulsed light beam, the fluorescent signal light can be reconstructed as two-dimensional information by the restoring section.

According to the scanning microscope 8 of this embodiment, even when signals at sites are adversely affected by scattering of the subject as a result of increasing the signal frequency, the correspondence relationship between pulsed beams and fluorescent signal light beams can be grasped easily through synchronization. Therefore, imaging can be performed at high speed and with high resolving power.

As described above, according to the scanning microscope 8 of this embodiment, temporal multiplexing and spatial multiplexing can be accomplished at the same time by converging, on one position, a plurality of pulsed beams having different optical path lengths and endowed with a relative angle by using the beam splitter apparatus 3. In this scanning microscope 8, parallel pulsed beams can be radiated onto different positions on the subject by spatial multiplexing. Furthermore, even when parallel pulsed beams are radiated, fluorescent signal light beams returning from the subject can be synchronized with the parallel pulsed beams through temporal multiplexing and can be separated from one another. For this reason, a decrease in resolving power as a result of radiating a plurality of pulsed beams at one time can be prevented, and therefore, fast scanning can be accomplished.

Although this embodiment has been described by way of an example where the beam splitter apparatus 3 according to the second embodiment is applied to a scanning microscope, the same effect can be brought about by applying a beam splitter apparatus according to another embodiment.

### Eighth Embodiment

A beam splitter apparatus 200 according to an eighth embodiment of the present invention will now be described with reference to the drawings.

For a description of this embodiment, the structures that are the same as those of the beam splitter apparatus 3 according to the above-described second embodiment are denoted by the same reference numerals, and thus a description thereof is omitted.

The beam splitter apparatus 200 according to this embodiment differs from the beam splitter apparatus 3 according to the above-described second embodiment in the incidence direction of pulsed beams from the pulsed light source 11 and the installation angles of the beam splitters 33 and 34. The other structures are the same as in the beam splitter apparatus 3 according to the second embodiment.

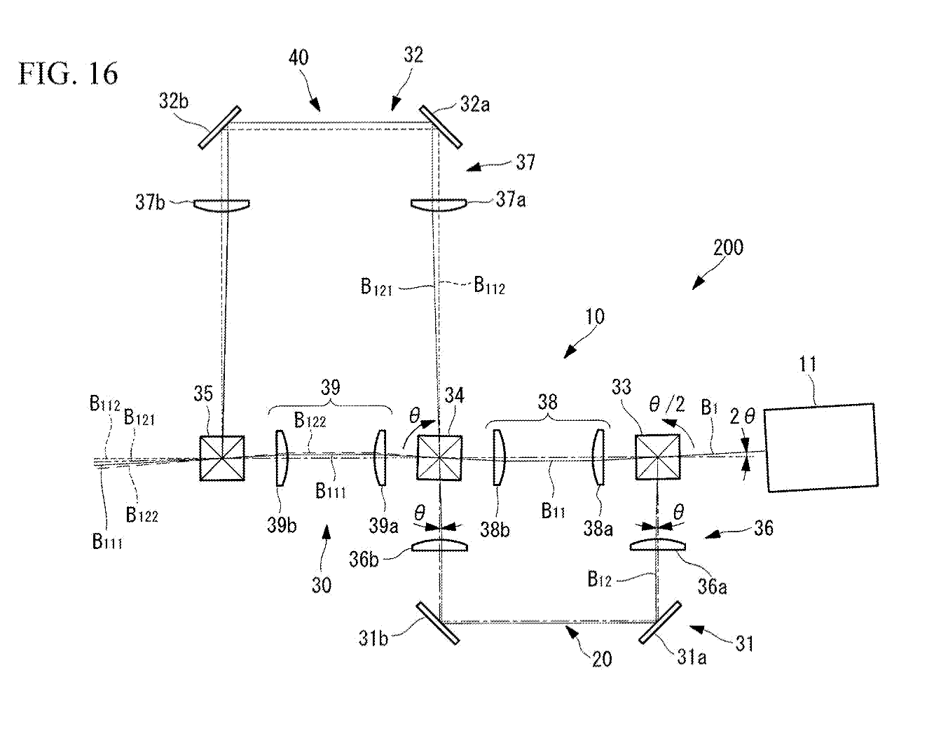

More specifically, in the beam splitter apparatus 200 according to this embodiment, as shown in FIG. 16, the propagation direction of a pulsed beam B₁ that is emitted from the pulsed light source 11 and is incident upon the beam splitter 33 is deflected in one direction (counterclockwise in the drawing) by an angle of 2θ relative to an extension (indicated by broken lines in the drawing) of a straight line connecting the centers of the beam splitters 33 and 34. Furthermore, in this embodiment, the installation angle of the beam splitter 33 is rotated in the same direction as above by an angle of θ/2, and the installation angle of the beam splitter 34 is rotated in the opposite direction to that described above (clockwise) by an angle of θ.

As a result, the incident angle of the pulsed beam B₁ upon the reflection surface of the beam splitter 33 is increased counterclockwise by an angle of 1.5θ, compared with the case of the beam splitter apparatus 3 according to the second embodiment. Therefore, the propagation direction of a pulsed beam B₁₂ reflected by the beam splitter 33 is tilted clockwise by an angle of θ relative to the propagation direction (indicated by broken lines in the drawing) of a pulsed beam in the second embodiment.

On the other hand, the propagation direction of a pulsed beam B₁₁ passing through the beam splitter 33 is set on an extension of the incidence pulsed beam B₁, regardless of the installation angle of the beam splitter 33. For the pulsed beam B₁₁ entering the optical path 10, its tilting direction is inverted by the relay optical system 38 composed of one pair of lenses 38a and 38b, and it is tilted clockwise by an angle of 2θ and is incident upon the beam splitter 34.

For the pulsed beam B₁₂ entering the optical path 20, its tilting direction is inverted via the relay optical system 36 composed of one pair of lenses 36a and 36b and the reflection optical system 31 including one pair of mirrors 31a and 31b. As a result, the pulsed beam B₁₂ is incident upon the beam splitter 34 at a counterclockwise angle of θ relative to the propagation direction (indicated by broken lines in the drawing) of the pulsed beam in the second embodiment.

The pulsed beams B₁₁ and B₁₂ are each branched into two at the beam splitter 34. The pulsed beam B₁₁ that is incident upon the beam splitter 34 with an angle of 2θ is incident upon the reflection surface of the beam splitter 34, which is tilted clockwise by an angle of θ, at an incident angle increased by θ clockwise compared with the case of the beam splitter apparatus 3 according to the second embodiment. Therefore, the propagation direction of a pulsed beam B₁₁₂ that is reflected at the beam splitter 34 and enters the optical path 40 coincides with the propagation direction of the pulsed beam in the second embodiment.

Furthermore, the pulsed beam B₁₂ that is incident upon the beam splitter 34 with an angle of θ is incident upon the reflection surface of the beam splitter 34, tilted clockwise by an angle of θ, at an incident angle increased by 2θ clockwise compared with the case of the beam splitter apparatus 3 according to the second embodiment. Therefore, the propagation direction of a pulsed beam B₁₂₂ that is reflected at the beam splitter 34 and enters the optical path 30 is tilted clockwise by an angle of 3θ relative to the propagation direction (indicated by broken lines in the drawing) of the pulsed beam in the second embodiment.

On the other hand, the propagation directions of pulsed beams B₁₁₁ and B₁₂₁ passing through the beam splitter 34 are set on extensions of the incident pulsed beams B₁₁ and B₁₂, regardless of the installation angle of the beam splitter 34.

For the pulsed beam B₁₂₁ in the optical path 40, its tilting direction is inverted via the relay optical system 37 composed of one pair of lenses 37a and 37b and the reflection optical system 32 composed of one pair of mirrors 32a and 32b. Because the pulsed beam B₁₁₂ is not tilted, the tilt angle does not change even after it has passed through the relay optical system 37 and the reflection optical system 32.

Furthermore, for pulsed beams B₁₁₁ and B₁₂₂ entering the optical path 30, their tilting directions are inverted via the relay optical system 39 composed of one pair of lenses 39a and 39b.

More specifically, the pulsed beams B₁₁₂ and B₁₂₁, which are tilted clockwise by angles of 0° and θ relative to the incidence axis tilted by 45° relative to the reflection surface, are incident upon the beam splitter 35 and are emitted in a direction tilted counterclockwise by angles of 0° and θ relative to the emission axis which is tilted by 45° relative to the reflection surface. Furthermore, the beam splitter 35 transmits the pulsed beams B₁₁₁ and B₁₂₂, which are tilted counterclockwise by angles 2θ and 3θ relative to a straight line connecting the beam splitters 34 and 35, without changing the tilt angles.

As a result, the four pulsed beams B₁₁₂, B₁₂₁, B₁₁₁, and B₁₂₂, endowed with time delays that are different from one another by the two optical paths (delay optical paths) 20 and 40 and spaced apart at the same angular interval of θ, are emitted from the beam splitter 35.

In this case, according to the beam splitter apparatus of this embodiment, for the pulsed beams B₁₂, B₁₂₁, and B₁₁₂ passing along the delay optical paths 20 and 40 provided with the relay optical systems 36 and 37 and the reflection optical systems 31 and 32, the tilt angles of their propagation directions can be controlled to an angle of θ or less. Therefore, lenses with a small aperture size can be employed as the lenses 36a, 36b, 37a, and 37b. This is advantageous in preventing an increase in apparatus size.

### Ninth Embodiment

A beam splitter apparatus 201 according to a ninth embodiment of the present invention will now be described with reference to the drawings.

For a description of this embodiment, the structures that are the same as those of the beam splitter apparatus 3 according to the above-described second embodiment are denoted by the same reference numerals, and thus a description thereof is omitted.

The beam splitter apparatus 201 according to this embodiment differs from the beam splitter apparatus 3 according to the above-described second embodiment in the installation angles of the beam splitters 34 and 35. The other structures are the same as in the beam splitter apparatus 3 according to the second embodiment.

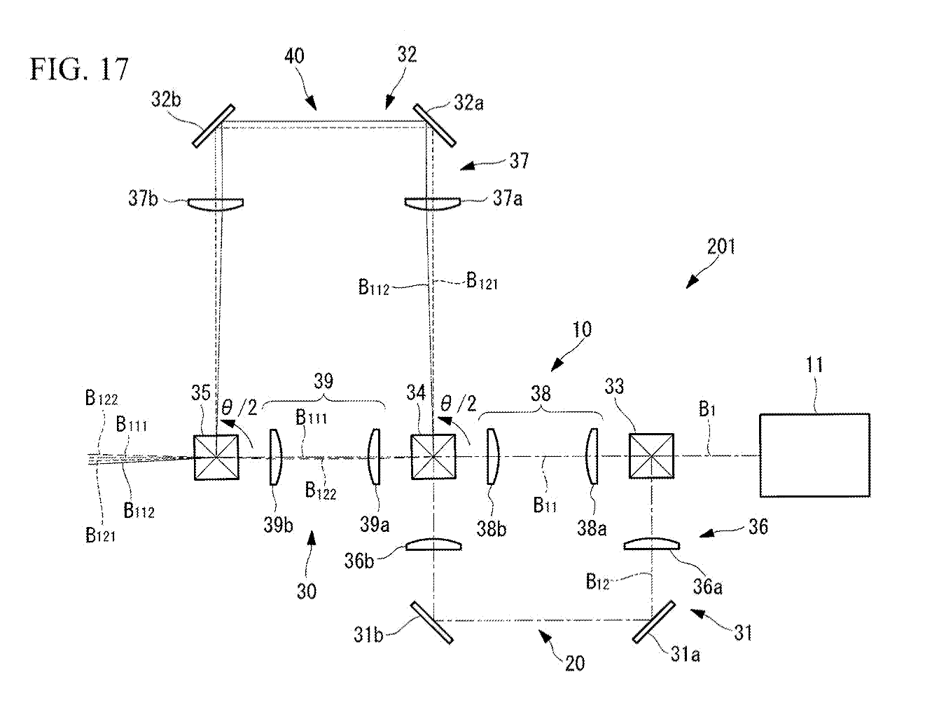

More specifically, in the beam splitter apparatus 201 according to this embodiment, as shown in FIG. 17, the installation angles of the beam splitters 34 and 35 are tilted in one direction (counterclockwise in the drawing) by an angle of θ/2 relative to the beam splitters 34 and 35 of the beam splitter apparatus 3 according to the second embodiment.

By doing so, the pulsed beams B₁₁ and B₁₂ passing along the optical paths up to the beam splitter 34 propagate along an optical axis with a tilt angle of 0°, as in the beam splitter apparatus 3 according to the second embodiment.

On the other hand, the pulsed beam B₁₁ incident upon the beam splitter 34 is branched into the pulsed beam B₁₁₁ that passes through it as-is with a tilt angle of 0° and the pulsed beam B₁₁₂ that is tilted counterclockwise by an angle of θ relative to a direction orthogonal to its direction. Furthermore, the pulsed beam B₁₂ incident upon the beam splitter 34 is branched into the pulsed beam B₁₂₁ that passes through it as-is with a tilt angle of 0° and the pulsed beam B₁₂₂ that is tilted counterclockwise by an angle of θ relative to a direction orthogonal to its direction.

The pulsed beam B₁₁₂ tilted counterclockwise by an angle of θ is incident upon the beam splitter 35 with its tilting direction inverted clockwise via the relay optical system 37 composed of the pair of lenses 37a and 37b and the reflection optical system 32 composed of the pair of mirrors 32a and 32b. Furthermore, the pulsed beam B₁₂₂ is incident upon the beam splitter 35 with its tilting direction inverted clockwise via the relay optical system 39 composed of the pair of lenses 39a and 39b.

Because the beam splitter 35 is tilted counterclockwise by an angle of θ/2, the pulsed beams B₁₁₂ and B₁₂₁ reflected by the reflection surface of this beam splitter 35 are emitted from the beam splitter 35 in directions tilted counterclockwise by angles of 2θ and θ, respectively. On the other hand, the pulsed beams B₁₁₁ and B₁₂₂ pass through the beam splitter 35 as-is and are emitted with a tilt angle of 0° in a direction tilted clockwise by an angle of θ.

As a result, the four pulsed beams B₁₁₂, B₁₂₁, B₁₁₁, and B₁₂₂ endowed with time delays that are different from one another by the two delay optical paths 20 and 40 and spaced apart at the same angular interval of θ are emitted from the beam splitter 35.

In this case, according to the beam splitter apparatus of this embodiment, for the pulsed beams B₁₁, B₁₂, B₁₁₁, B₁₁₂, B₁₂₁, and B₁₂₂ passing along not just the delay optical paths but all optical paths, the tilt angles of their propagation directions can be controlled to an angle of θ. Therefore, lenses with a small aperture size can be employed as the lenses 36a, 36b, 37a, 37b, 38a, 38b, 39a, and 39b. This is advantageous in preventing an increase in apparatus size.

### Tenth Embodiment

A beam splitter apparatus 202 according to a tenth embodiment of the present invention will now be described with reference to the drawings.

For a description of this embodiment, the structures that are the same as those of the beam splitter apparatus 3 according to the above-described second embodiment are denoted by the same reference numerals, and thus a description thereof is omitted.

The beam splitter apparatus 202 according to this embodiment differs from the beam splitter apparatus 3 according to the above-described second embodiment in the incidence direction of the pulsed beam B₁ from the pulsed light source 11 and the installation angles of the beam splitters 33 and 34. The other structures are the same as in the beam splitter apparatus 3 according to the second embodiment.

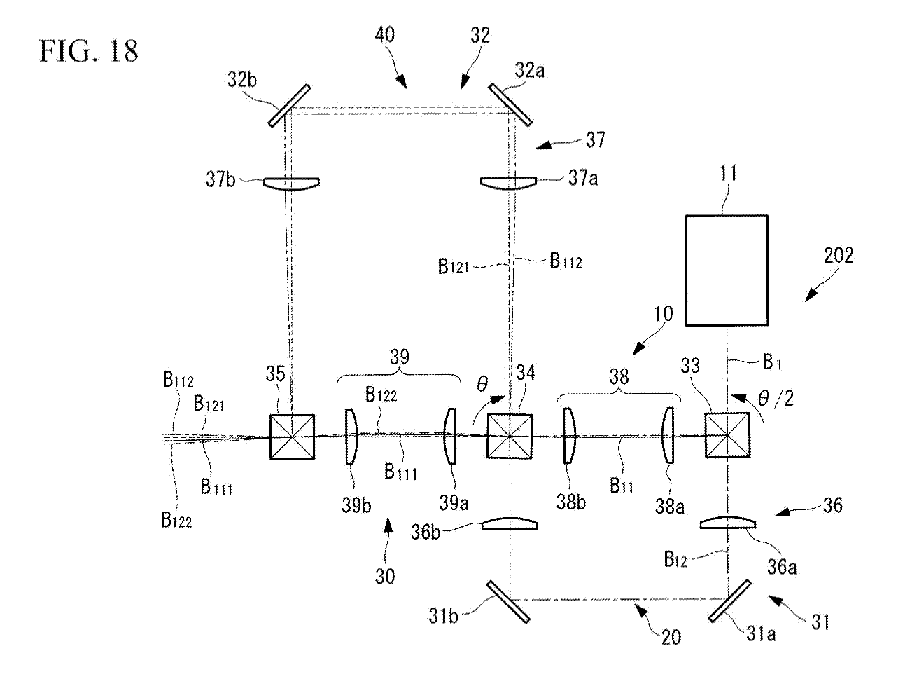

More specifically, in the beam splitter apparatus 202 according to this embodiment, as shown in FIG. 18, the incidence direction of the pulsed beam B₁ from the pulsed light source 11 to the beam splitter 33 is set in a direction orthogonal to a straight line connecting the beam splitters 33 and 34.

Furthermore, the installation angle of the beam splitter 33 is tilted in one direction (counterclockwise in the drawing) by an angle of θ/2 relative to the beam splitter 33 of the beam splitter apparatus 3 according to the second embodiment. Furthermore, the installation angle of the beam splitter 34 is tilted in the opposite direction to the rotation of this beam splitter 33 (clockwise in the drawing) by an angle of θ relative to the beam splitter 34 of the beam splitter apparatus 3 according to the second embodiment.

By doing so, the pulsed beam B₁₂ that enters the delay optical path 20 through the beam splitter 33 propagates along an optical axis with a tilt angle of 0°, as in the beam splitter apparatus 3 according to the second embodiment.

On the other hand, the pulsed beam B₁₁ reflected at the beam splitter 33 is tilted counterclockwise by an angle of θ relative to a straight line connecting the beam splitters 33 and 34.

The pulsed beam B₁ is incident upon the beam splitter 34 after its tilting direction has been inverted via the relay optical system 38 composed of the pair of lenses 38a and 38b. The pulsed beam B₁₁ incident upon the beam splitter 34 is branched into the pulsed beam B₁₁₁ passing through it as-is with a tilt angle of θ and the pulsed beam B₁₁₂ tilted clockwise by an angle of θ relative to a direction orthogonal to its direction.

Furthermore, the pulsed beam B₁₂ incident upon the beam splitter 34 is branched into the pulsed beam B₁₂₁ passing through it as-is with a tilt angle of 0° and the pulsed beam B₁₂₂ tilted clockwise by an angle of 2θ relative to a direction orthogonal to its direction.

The pulsed beam B₁₁₂ tilted clockwise by an angle of θ is incident upon the beam splitter 35 with its tilting direction inverted counterclockwise via the relay optical system 37 composed of the pair of lenses 37a and 37b and the reflection optical system 32 composed of the pair of mirrors 32a and 32b. Furthermore, the pulsed beams B₁₁₁ and B₁₂₂ are incident upon the beam splitter 35 with their tilting directions inverted counterclockwise via the relay optical system 39 composed of the pair of lenses 39a and 39b.

The pulsed beams B₁₁₂ and B₁₂₁ reflected by the reflection surface of the beam splitter 35 are emitted from the beam splitter 35 in directions tilted clockwise by an angle of θ and clockwise by an angle of 0°. On the other hand, the pulsed beams B₁₁₁ and B₁₂₂ pass through the beam splitter 35 as-is and are emitted in directions tilted counterclockwise by a tilt angle of θ and a tilt angle of 2θ.

As a result, the four pulsed beams B₁₁₂, B₁₂₁, B₁₁₁, and B₁₂₂ endowed with time delays that are different from one another by the two delay optical paths 20 and 40 and spaced apart at the same angular interval of θ are emitted from the beam splitter 35.

In this case, according to the beam splitter apparatus 202 of this embodiment, for the pulsed beams B₁₂, B₁₂₁, and B₁₁₂ passing along the delay optical paths 20 and 40 provided with the relay optical systems 36 and 37 and the reflection optical systems 31 and 32, the tilt angles of their propagation directions can be controlled to an angle of θ or less. Therefore, lenses with a small aperture size can be employed as the lenses 36a, 36b, 37a, and 37b. This is advantageous in preventing an increase in apparatus size. The last branching means in this embodiment may be formed of a polarizing beam splitter.

### Eleventh Embodiment

A beam splitter apparatus 203 according to an eleventh embodiment of the present invention will now be described with reference to the drawings.

For a description of this embodiment, the structures that are the same as those of the beam splitter apparatus 3 according to the above-described second embodiment are denoted by the same reference numerals, and thus a description thereof is omitted.

The beam splitter apparatus 203 according to this embodiment differs from the beam splitter apparatus 3 according to the above-described second embodiment in the incident positions of pulsed beams from the delay optical paths 20 and 40 upon the beam splitters 34 and 35 and in relay optical systems 104 and 105.

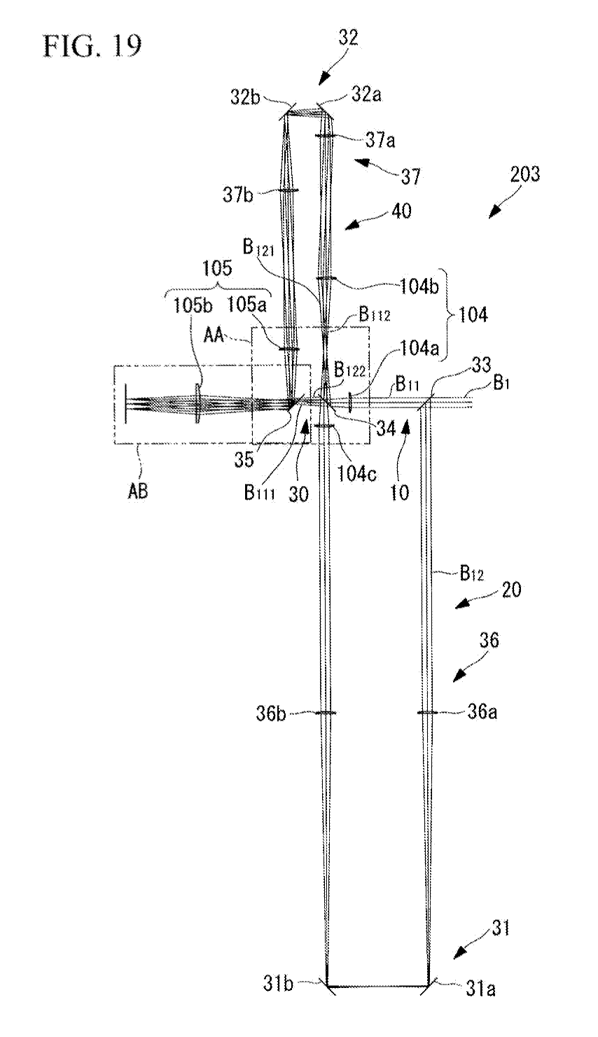

As shown in FIG. 19, the beam splitter apparatus 203 according to this embodiment includes a relay optical system 104 composed of lenses 104a, 104b, and 104c that relay the pupils of the pulsed beams B₁₁₂ and B₁₂₁, entering the optical path 40, originating from the pulsed beams B₁₁ and B₁₂ propagating along the optical paths 10 and 20 branching off from each other via the beam splitter 33; and a relay optical system 105 composed of lenses 105a and 105b that relay the pupils of the pulsed beams B₁₁₂ and B₁₂₁ from the optical path 40 before and after the beam splitter 35.

Furthermore, the lenses 104a and 105b constitute a relay optical system that relays the pupils of the pulsed beams B₁₁₁ and B₁₂₂ passing through the beam splitters 34 and 35.

More specifically, the pulsed beam B₁ incident upon the beam splitter 33 as a collimated beam is branched by the beam splitter 33 into the pulsed beams B₁₁ and B₁₂ composed of two collimated beams.

The pulsed beam B₁₁ composed of a collimated beam is collected by the lens 104a and is partly reflected by the beam splitter 34. The reflected portion of the beam B₁₁ enters the delay optical path 40 as the pulsed beam B₁₁₂. In the delay optical path 40, the pulsed beam B₁₁₂ is converted by the lens 104b into the pulsed beam B₁₁₂ composed of a collimated beam.

Then, it is converted into a collimated beam via the relay optical system 37 and the reflection optical system 32, is collected by the lens 105a, is reflected by the beam splitter 35, and is emitted by the lens 105b in the form of a collimated beam again.

The pulsed beam B₁₁₁ passing through the beam splitters 34 and 35 is emitted by the lens 105b in the form of a collimated beam again.

On the other hand, the pulsed beam B₁₂ composed of a collimated beam is introduced into the delay optical path 20, is converted into a collimated beam via the relay optical system 36 and the reflection optical system 31, is collected by the lens 104c, and is incident upon the beam splitter 34. The pulsed beam B₁₂ is branched into the pulsed beams B₁₂₁ and B₁₂₂ at the beam splitter 34, and the pulsed beam B₁₂₁ passing through the beam splitter 34 is emitted from the lens 105b in the form of a collimated beam while its pupil is being relayed, like the pulsed beam B₁₁₁.

Furthermore, the pulsed beam B₁₂₂ reflected at the beam splitter 34 is emitted from the lens 105b in the form of a collimated beam while its pupil is being relayed, like the pulsed beam B₁₁₁.

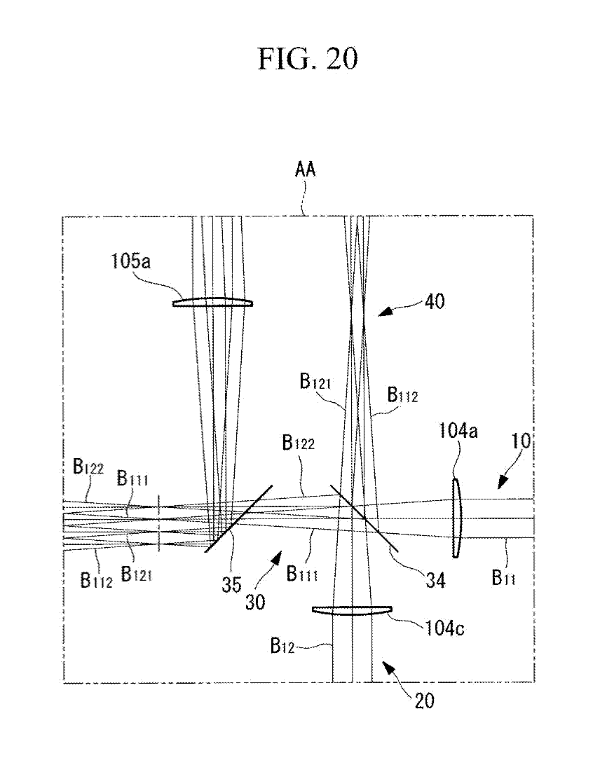

In this case, in this embodiment, as shown in FIG. 20, the optical axes of the pulsed beams B₁₁ and B₁₂ incident upon the beam splitter 34 are shifted so as not to coincide on the reflection surface of the beam splitter 34 by adjusting the positions of the reflection optical system 31 and the relay optical system 36. Furthermore, the optical axes of the splitter 35 are shifted apart at regular intervals on the reflection surface by adjusting the positions of the reflection optical system 32 and the relay optical system 37. FIG. 20 is a magnified view of area AA in FIG. 19.

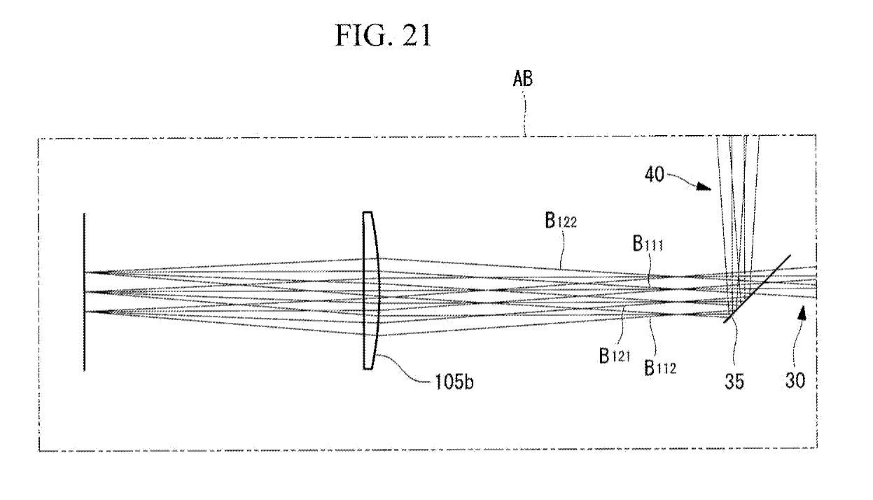

Then, the principal rays of the pulsed beams B₁₁₁, B₁₁₂, B₁₂₁, and B₁₂₂ multiplexed by the beam splitter 35 are set to become parallel to one another. Furthermore, as shown in FIG. 21, the pulsed beams B₁₁₁, B₁₁₂, B₁₂₁, and B₁₂₂ multiplexed by the beam splitter 35 are set to be collected on the same flat surface after passing through the beam splitter 35. By doing so, the lens 105b works as a telecentric optical system for the pulsed beams B₁₁₁, B₁₁₂, B₁₂₁, and B₁₂₂, and the pulsed beams B₁₁₁, B₁₁₂, B₁₂₁, and B₁₂₂ are made to have different angles by the lens 105b and converged on the same position at the back focal position of the lens 105b. FIG. 21 is a magnified view of area AB of FIG. 19.

In other words, the four pulsed beams B₁₁₁, B₁₁₂, B₁₂₁, and B₁₂₂, endowed with time delays different from one another by the two delay optical paths 20 and 40 and made to have different angles, are emitted from the back focal position of the lens 105b.

This brings an advantage in that when the pulsed beams B₁₁₁, B₁₁₂, B₁₂₁, and B₁₂₂ are collected by the subsequent objective lens at different sites spaced apart on the subject to generate fluorescence, the generated fluorescence can be prevented from being mixed and observation with high spatial resolving power can be accomplished because the light beams B₁₁₁, B₁₁₂, B₁₂₁, and B₁₂₂ are endowed with different time delays from one another.

In this embodiment, the optical path length may be adjusted and the intervals between the optical axes of the pulsed beams B₁₁₁, B₁₁₂, B₁₂₁, and B₁₂₂ incident upon the lens 105b may be adjusted by rectilinearly translating at least one of the mirrors 31a and 31b disposed in the delay optical path 20 and at least one of the mirrors 32a and 32b disposed in the delay optical path 40, for example, the mirrors 31b and 32b, relative to the other mirrors 31a and 32a on a plane parallel to the optical axis between the mirrors 31a and 31b or the mirrors 32a and 32b.

Furthermore, the reflection optical systems 31 and 32 may be rectilinearly translated in a direction along the optical axis between the mirrors 31a and 31b; 32a and 32b. By doing so, the intervals between the optical axes of the pulsed beams B₁₁₁, B₁₁₂, B₁₂₁, and B₁₂₂ incident upon the lens 105b can be adjusted without having to change the optical path length. Therefore, this brings an advantage in that it is not necessary to re-adjust the optical path length.

Furthermore, if the optical axes are shifted by moving the mirrors 31b and 32b of the reflection optical systems 31 and 32, it is preferable that the lenses 36b and 104c and 37b and 105a be moved in a direction orthogonal to the optical axes by the same amounts as the displacement of the optical axes. This brings an advantage in that the principal rays of the pulsed beams B₁₁₁, B₁₁₂, B₁₂₁, and B₁₂₂, after being multiplexed by the beam splitter 35, can be maintained parallel to prevent the point of convergence from being shifted in the optical-axis direction.

Furthermore, in this embodiment, the beam diameters of the pulsed beams B₁₁₁, B₁₁₂, B₁₂₁, and B₁₂₂ can be made the same by relaying a pupil with the plurality of relay optical systems 36, 37, 104, and 105. This provides an advantage in that because the beam diameters are not changed, the resolving power can be prevented from changing when this embodiment is applied to a scanning observation apparatus. Furthermore, the lenses 36a 36b, 37a, 37b, 104a, 104b, 104c, and 105a disposed in the optical paths 10, 20, 30, and 40 may be set to have the same focal length.

In addition, a polarizing beam splitter may be employed as the beam splitters 33, 34, and 35. By doing so, pulsed beams can be used without loss.

Furthermore, in this embodiment, because the optical axes of the pulsed beams B₁₁₁, B₁₁₂, B₁₂₁, and B₁₂₂ propagating along the optical paths 10, 20, 30, and 40 are arranged at regular intervals as a result of the multiplexing operation, the scanning pitches of the pulsed beams B₁₁₁, B₁₁₂, B₁₂₁, and B₁₂₂ on the subject can be made uniform to allow images free of nonuniform resolving power to be acquired when this embodiment is applied to a scanning observation apparatus.

Furthermore, when this embodiment is to be applied to a scanning observation apparatus, it is preferable that the position of convergence of the pulsed beams B₁₁₁, B₁₁₂, B₁₂₁, and B₁₂₂ or a position that is optically conjugate to it be disposed on the swing axis of the scanner. This brings an advantage in that even when the scanner is swung to scan a pulsed beam, the incident position of the pulsed beam upon the scanner does not change and the pupil is maintained intact, allowing the scanning area to be scanned without omission.

Furthermore, in the case where the scanner is a raster scanning scanner, it is preferable that the position of convergence of pulsed beams or a position that is optically conjugate to it be disposed on the swing axis of the slower scanner. This brings an advantage in that scanning is completed in a short time without having to increase the scanning frequency of the faster scanner because the scanning area is divided by producing a plurality of pulsed beams.

### Twelfth Embodiment

A beam splitter apparatus 204 according to a twelfth embodiment of the present invention will now be described with reference to the drawings.

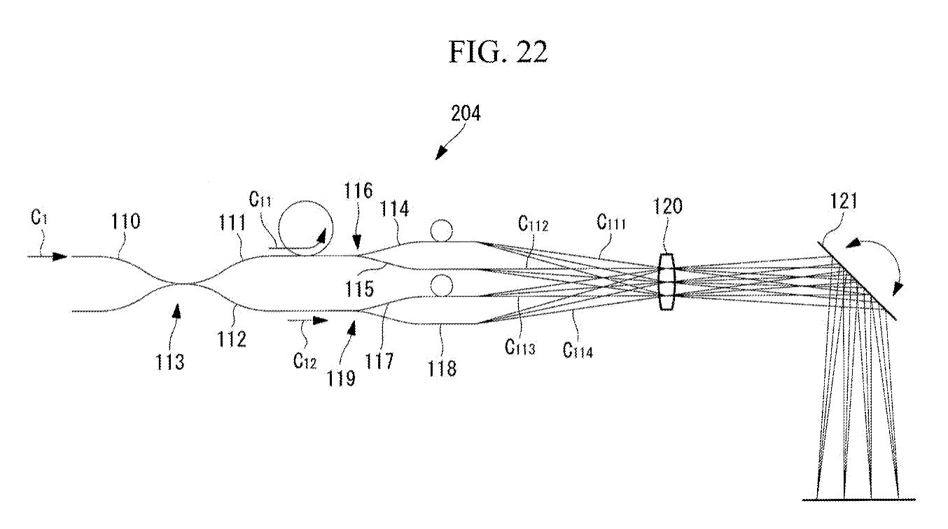

As shown in FIG. 22, the beam splitter apparatus 204 according to this embodiment includes an optical fiber 110 that guides a pulsed beam C₁ emitted from a light source; a fiber coupler 113 that branches the pulsed beam C₁ propagating in the optical fiber 110 into pulsed beams C₁₁ and C₁₂ propagating in optical fibers 111 and 112; a fiber coupler 116 that branches the pulsed beam C₁₁ propagating in the optical fiber 111 into optical fibers 114 and 115; and a fiber coupler 119 that branches the pulsed beam C₁₂ propagating in the optical fiber 112 into optical fibers 117 and 118. Four pulsed beams C₁₁₁, C₁₁₂, C₁₁₃, and C₁₁₄ emitted from the ends of the four optical fibers 114, 115, 117, and 118 are endowed with relative angles by adjusting the end angles of the optical fibers 114 and 115, 117, 118 (beam-angle setting section) and are converged on the same position.

One set of the optical fibers 111, 114, and 117 branching off via the three fiber couplers 113, 116, and 119, respectively, is longer than another set of the optical fibers 112, 115, and 118, so that the lengths of the optical paths along which the four pulsed beams C₁₁₁, C₁₁₂, C₁₁₃, and C₁₁₄ propagate until they are emitted from the ends of the optical fibers 114, 115, 117, and 118 are made different from each other. In FIG. 22, reference numeral 120 denotes a focusing lens that collects the pulsed beams C₁₁₁, C₁₁₂, C₁₁₃, and C₁₁₄ converged on the same position by the optical fibers 114, 115, 117, and 118 and forms images of the exit ends of the optical fibers 114, 115, 117, and 118 on the subject. Reference numeral 121 denotes a scanner that scans the subject with the pulsed beams C₁₁₁, C₁₁₂, C₁₁₃, and C₁₁₄.

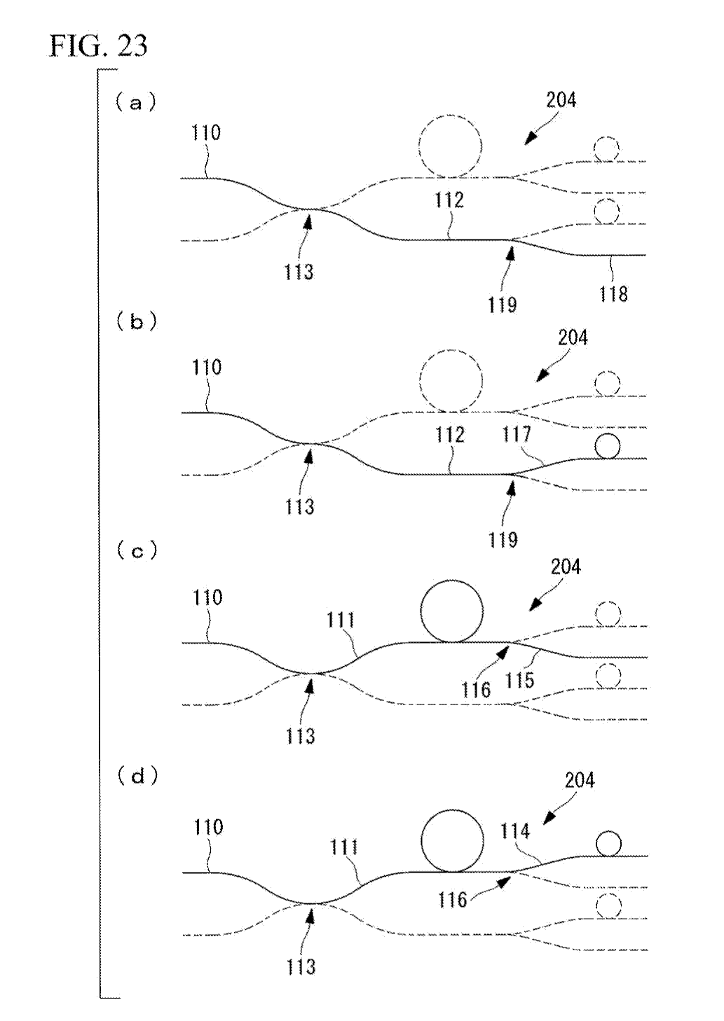

FIG. 23(a) shows a path with the shortest optical path length from the optical fiber 110 to the exit port of the optical fiber 118 via the two fiber couplers 113 and 119. FIG. 23(b) shows a path with the second-shortest optical path length from the optical fiber 110 to the exit end of the optical fiber 117 via the two fiber couplers 113 and 119. FIG. 23(c) denotes a path with the second-longest optical path length from the optical fiber 110 to the exit end of the optical fiber 115 via the two fiber coupler 113 and 116. FIG. 23(d) shows a path with the longest optical path length from the optical fiber 110 to the exit end of the optical fiber 114 via the two fiber couplers 113 and 116.

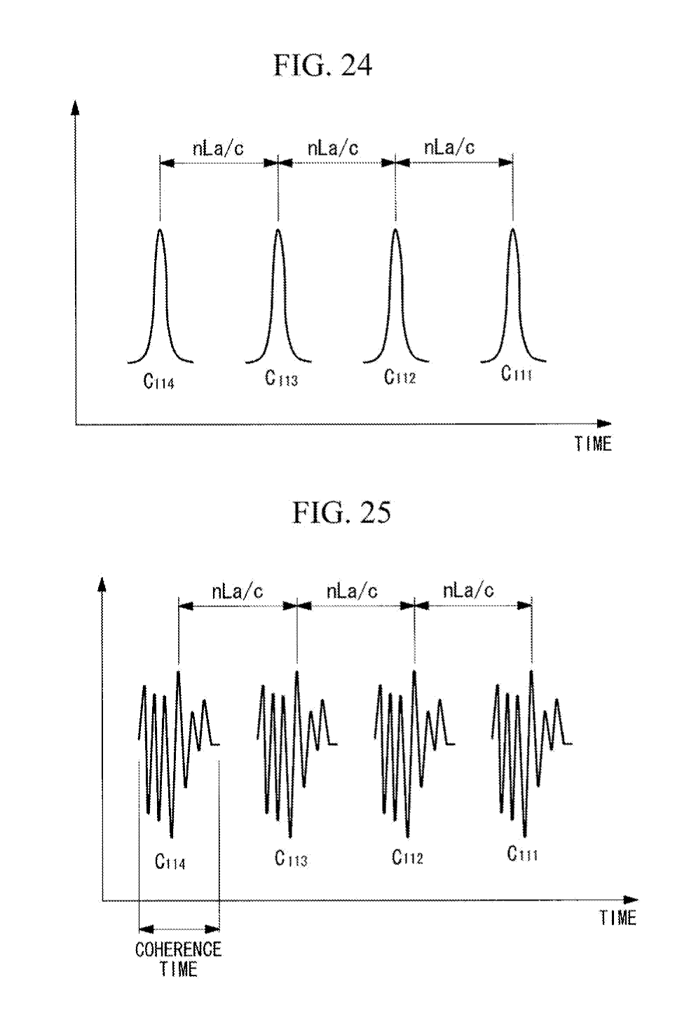

For example, if the difference in length between the optical fibers 111 and 112 is set as 2La and the differences between the optical fibers 114 and 115 and between 117 and 118 are set as La, the differences in path length from the shortest path are La, 2La, and 3La. Consequently, when the pulsed beam C₁ is incident upon the optical fiber 110, an optical pulse train with a time interval of nLa/c is generated, as shown in FIG. 24. Here, n indicates the refractive index of the cores of the optical fibers 110, 111, 112, 114, 115, 117, and 118, and c indicates the velocity of light, assuming that the spatial length converted from the pulse widths of the pulsed beams C₁₁₁, C₁₁₂, C₁₁₃, and C₁₁₄ is sufficiently small.

Then, with the beam splitter apparatus 204 of this embodiment having the above-described structure, there is an advantage in that when a light beam with small temporal coherence is emitted as the pulsed beam C₁, deterioration due to illumination interference can be prevented because the four pulsed beams C₁₁₁, C₁₁₂, C₁₁₃, and C₁₁₄ emitted with a time interval of nLa/c do not interfere with one another, as shown in FIG. 25.

In addition, the four pulsed beams C₁₁₁, C₁₁₂, C₁₁₃, and C₁₁₄ branching off in this manner are collected by the focusing lens 120 and are scanned by the scanner 121 over the subject, as shown in FIG. 22. The focusing lens 120 forms images of the exit ends of the optical fibers 114, 115, 117, and 118 on the subject via the scanner 121. As shown in FIG. 22, the scanner 121 is a mirror swung about an axis orthogonal to the drawing and can scan the pulsed beams C₁₁₁, C₁₁₂, C₁₁₃, C₁₁₄ in a direction parallel to the drawing while being swung.

By doing so, the time required to irradiate an area with pulsed beams can be reduced to one fourth of that when the same area is scanned with a single pulsed beam without spatial multiplexing. There is another advantage in that observed images can be acquired without being adversely affected by interference because delay times are provided among the pulsed beams C₁₁₁, C₁₁₂, C₁₁₃, and C₁₁₄ to enable temporal multiplexing.

In this embodiment, the following modification can be employed.

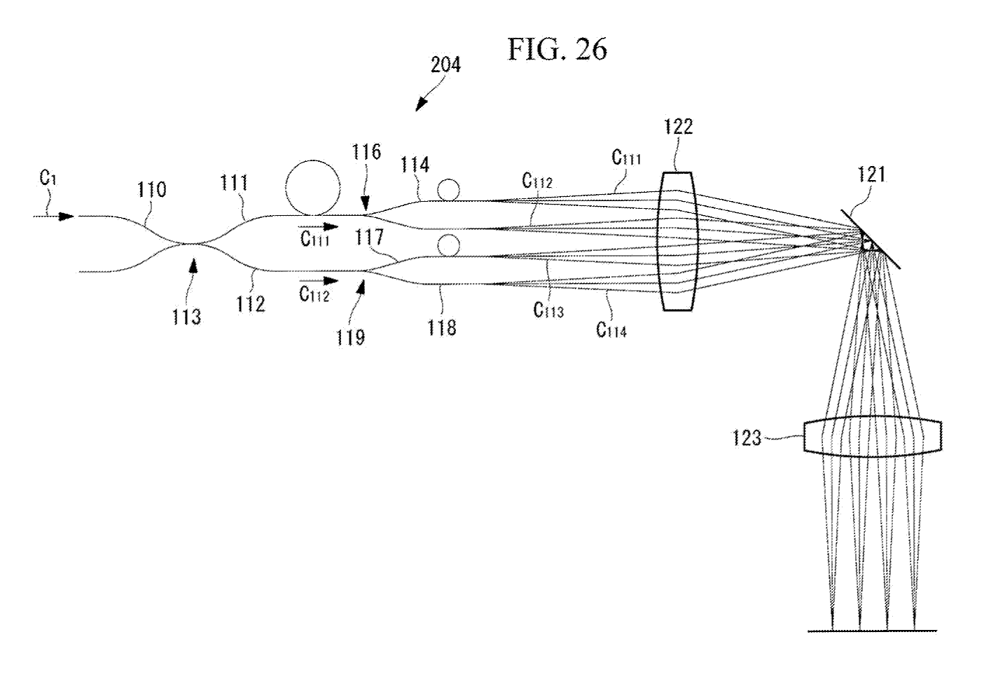

More specifically, two positive lenses 122 and 123 may be employed, as shown in FIG. 26, instead of collecting the four pulsed beams C₁₁₁, C₁₁₂, C₁₁₃, and C₁₁₄ with the single focusing lens 120. In this case, the exit ends of the optical fibers 114, 115, 117, and 118 are disposed near the front focal plane of the positive lens 122, the scanner 121 is disposed near the back focal plane of the positive lens 122, and furthermore, the scanner 121 is disposed near the front focal plane of the positive lens 123. By doing so, a telecentric arrangement can be achieved both on the object side and the image side, so that observation without a large change in the magnification can be accomplished even when the subject is moved back and forth on the optical axis.

Furthermore, although this embodiment has been described by way of an example where the one pulsed beam C₁ is branched into the four pulsed beams C₁₁₁, C₁₁₂, C₁₁₃, and C₁₁₄, the pulsed beam C₁ may be branched into any other number of pulsed beams.

Furthermore, although the above-described embodiment has discussed a member that performs one-dimensional scanning, such as a single galvanometer mirror, as the scanner 121, two-dimensional scanning may be performed by adding another scanner.

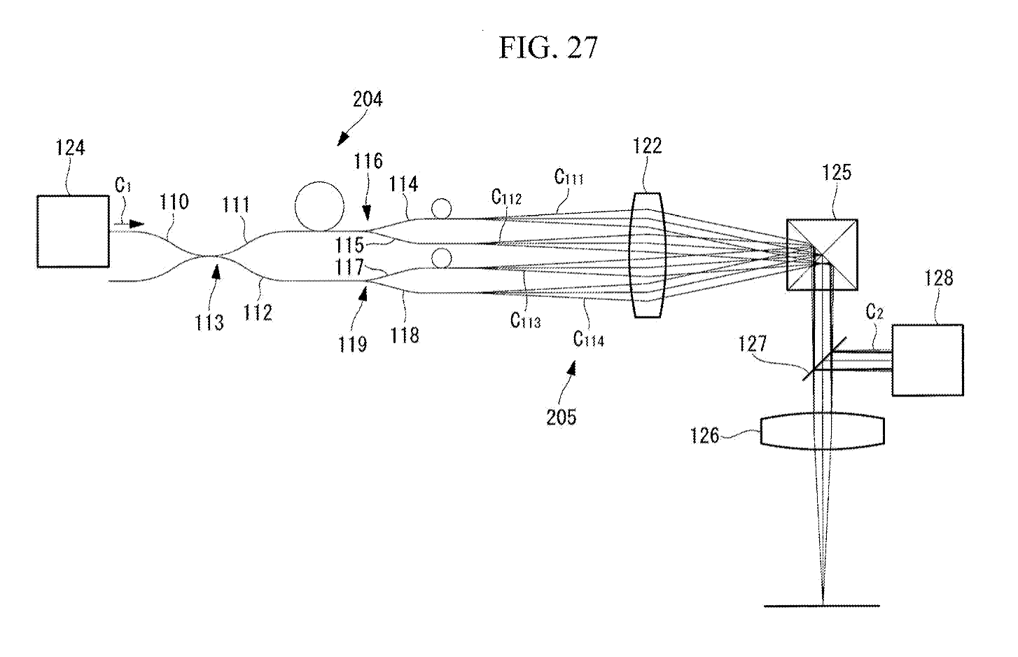

An example where this embodiment is applied to a fluoroscopy apparatus 205, as shown in FIG. 27, will be described. This fluoroscopy apparatus 205 includes the beam splitter apparatus 204 according to this embodiment; a pulsed light source 124 that produces the pulsed beam C₁ entering this beam splitter apparatus 204; a focusing lens 122 that collects the pulsed beams C₁₁₁, C₁₁₂, C₁₁₃, and C₁₁₄ emitted from the beam splitter apparatus 204; a scanner 125 provided with two galvanometer mirrors that can swing about axes intersecting each other; an objective lens 126 that focuses on the subject the pulsed beams C₁₁₁, C₁₁₂, C₁₁₃, and C₁₁₄ scanned by the scanner 125; a dichroic mirror 127 that branches fluorescence (return light) C₂ produced at the subject and collected by the objective lens 126 off from the optical paths of the pulsed beams C₁₁₁, C₁₁₂, C₁₁₃, and C₁₁₄; and an optical detector 128 that detects the fluorescence C₂ branching off via this dichroic mirror 127.

According to this fluoroscopy apparatus 205, after a light beam has been emitted from the pulsed light source 124 and branched into four light beams by the beam splitter apparatus 204, the resultant pulsed beams C₁₁₁, C₁₁₂, C₁₁₃, and C₁₁₄ scanned two-dimensionally by the scanner 125 are focused on the subject by the objective lens 126, so that the fluorescence C₂ can be produced at the subject. Thereafter, the fluorescence C₂ produced in the subject and collected by the objective lens 126 is branched by the dichroic mirror 127 off from the pulsed beams C₁₁₁, C₁₁₂, C₁₁₃, and C₁₁₄ so as to be detected by the optical detector 128. In this case, a two-dimensional fluorescence image can be acquired by storing the scanning position by the scanner 125 and the intensity of the fluorescence C₂ detected by the optical detector 128 in association with each other to perform fluoroscopy of the subject.

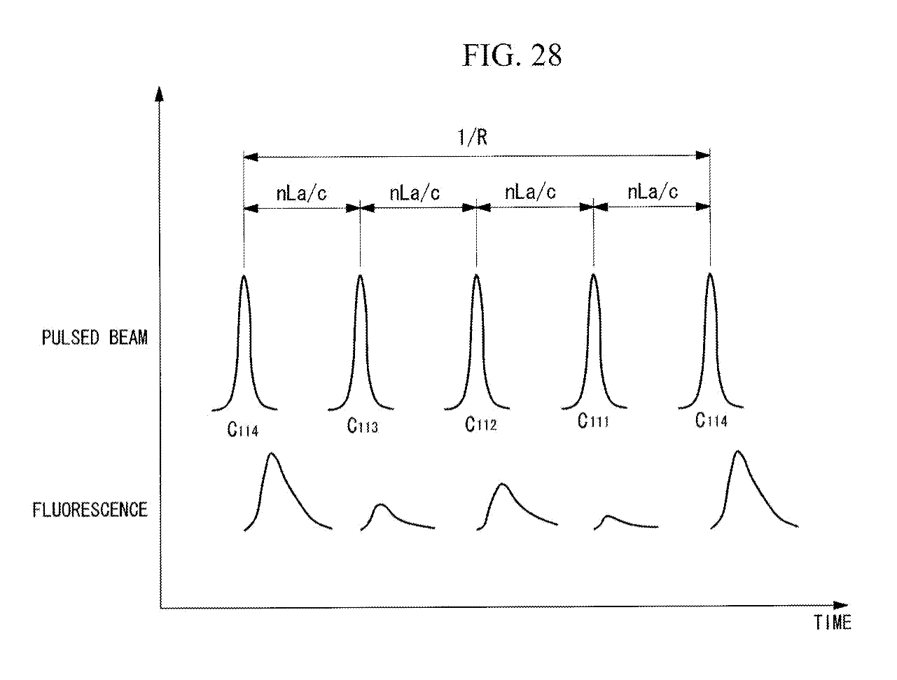

Because the pulsed beams C₁₁₁, C₁₁₂, C₁₁₃, and C₁₁₄ are multiplexed both spatially and temporally by the beam splitter apparatus 204, the acquired fluorescence C₂ forms a train of pulses that do not interfere with each other, as shown in FIG. 28, and if the optical detector 128, such as a photomultiplier tube having sufficiently high response speed, is used, four pulses of fluorescence C₂ can be detected by separating them in the time domain without having to employ a two-dimensional image pickup element.

Because the subject is irradiated with the four pulsed beams C₁₁₁, C₁₁₂, C₁₁₃, and C₁₁₄, processing can be performed at a speed four times as high as that of scanning based on the normal one-point-irradiation and one-point-detection technique. In short, even if the scanning speed of the scanner 125 is changed to one fourth of that of scanning based on the one-point-irradiation and one-point-detection technique, image acquisition with the same frame rate can be accomplished.

More specifically, when 1/R=4nLa/c is satisfied, where R is the repetition frequency of pulsed oscillation by the pulsed light source 124 and nLa/c is a pulse interval depending on the lengths of the optical fibers 114 and 115, 117, and 118, the pulsed beam C₁ oscillated from the pulsed light source 124 is multiplexed into four beams spaced apart at regular intervals, and a fluorescence C₂ pulse train produced by a line of the resultant pulsed beams C₁₁₁, C₁₁₂, C₁₁₃, and C₁₁₄ can be acquired with the same repetition period, as shown in FIG. 28.

### Thirteenth Embodiment

A beam splitter apparatus 206 according to a thirteenth embodiment of the present invention will now be described with reference to drawings.

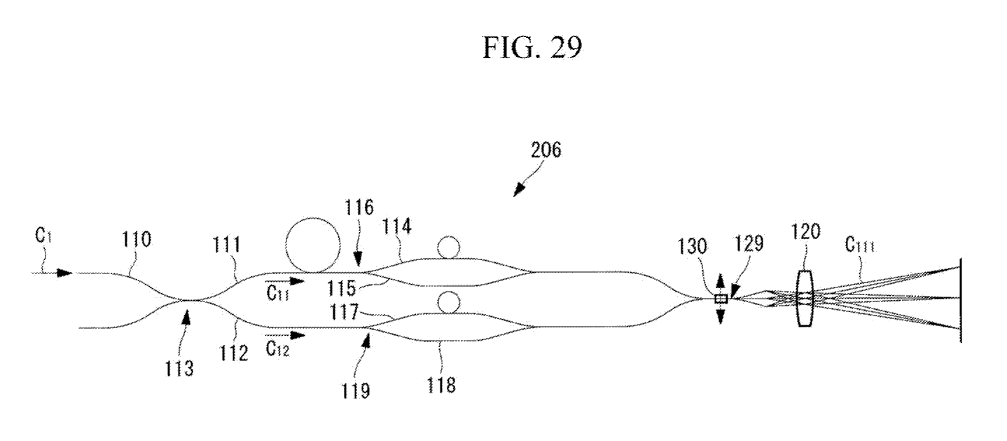

As shown in FIG. 29, in the beam splitter apparatus 206 according to this embodiment, the exit ends of the four optical fibers 114, 115, 117, and 118 in the beam splitter apparatus 204 according to the twelfth embodiment are bundled and a scanner 130 that shifts an optical fiber bundle 129 of the bundled fibers in the radial direction is provided.

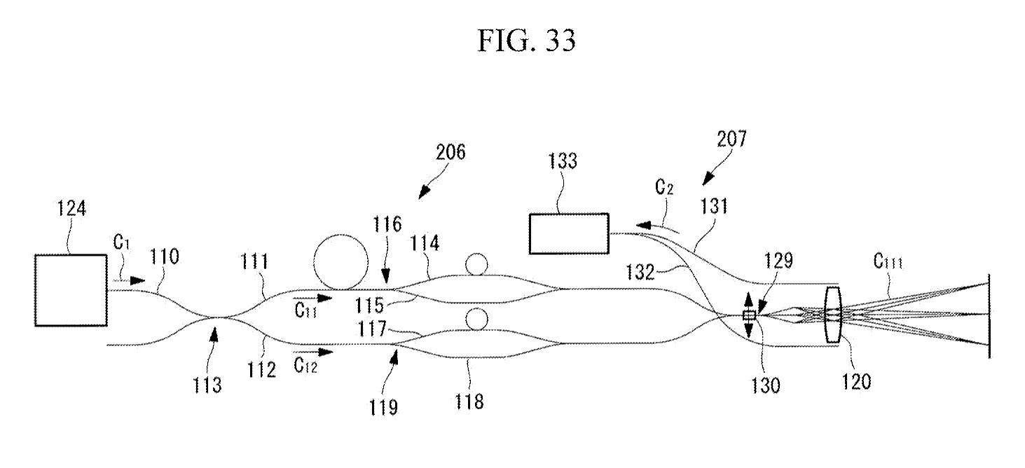

The scanner 130 can resonate the optical fiber bundle 129 one-dimensionally or two-dimensionally in the radial direction and can collect the pulsed beams C₁₁₁, C₁₁₂, C₁₁₃, and C₁₁₄ emitted from the exit ends of the optical fibers 114, 115, 117, and 118 by the focusing lens 120 disposed at the pupil positions to scan the subject disposed at positions that are optically conjugate to the exit ends. Although only the pulsed beam C₁₁₁ is shown in FIGS. 29 and 33, actually C₁₁₂, C₁₁₃, and C₁₁₄ are scanned near this C₁₁₁.

Unlike the twelfth embodiment, in which a mirror 121 is swung to scan the pulsed beams C₁₁₁, C₁₁₂, C₁₁₃, and C₁₁₄, the size can be reduced and adjustment can be simplified.

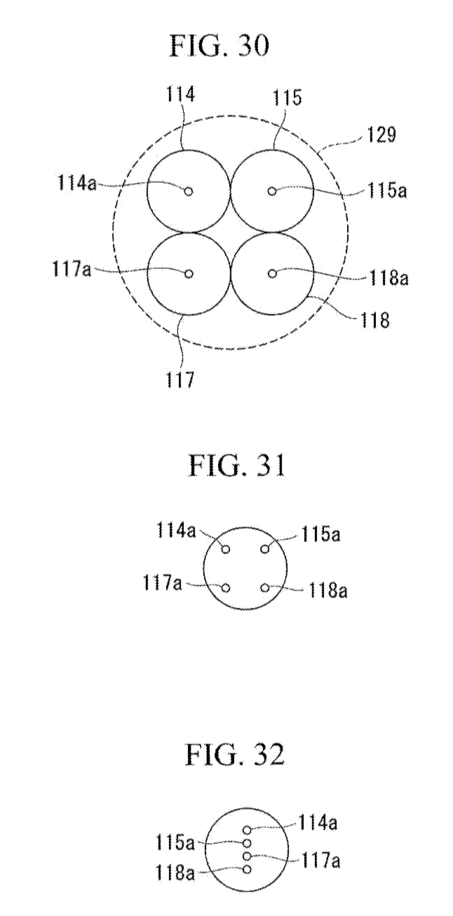

As shown in FIG. 30, in this embodiment, the exit ends of the four optical fibers 114, 115, 117, and 118 may be bundled so that all the optical fibers 114, 115, 117, and 118 are adjacent, or alternatively, the claddings of the four optical fibers 114, 115, 117, and 118 may be fused to arrange cores 114a, 115a, 117a, and 118a so that they are adjacent to one another. In this case, the cores 114a, 115a, 117a, and 118a may be arranged in a rectangular shape, as shown in FIG. 31, or in a line, as shown in FIG. 32.

The beam splitter apparatus 206 according to this embodiment with the above-described structure is provided in a fluoroscopy apparatus 207, as shown in FIG. 33. This beam splitter apparatus 206 splits the pulsed beam C₁ from the pulsed light source 124 connected to one end of the optical fiber 110 into the four pulsed beams C₁₁₁, C₁₁₂, C₁₁₃, and C₁₁₄, which are emitted from the exit ends and collected by an objective lens 120. By doing so, images of the exit ends of the optical fibers 114, 115, 117, and 118 can be formed on the subject disposed at positions that are optically conjugate to the exit ends of the optical fibers 114, 115, 117, and 118 to radiate four pulsed beams C₁₁₁, C₁₁₂, C₁₁₃, and C₁₁₄.

In FIG. 33, optical fibers 131 and 132 whose end portions are disposed around the objective lens 120 are provided. The fluorescence C₂ generated at the positions irradiated with the pulsed beam C₁₁₁, C₁₁₂, C₁₁₃, and C₁₁₄ on the subject is incident upon the end portions of the optical fibers 131 and 132, is guided in the optical fibers 131 and 132, and is detected by an optical detector 133 connected to the other ends of the optical fibers 131 and 132.

Although the fluorescence C₂ is guided in the two optical fibers 131 and 132 in FIG. 33, a space may be provided around the objective lens 120 to arrange the end portions of three or more optical fibers instead. As a result, fluorescence images with a high SN ratio can be acquired.

The present invention is not limited to the above described embodiment of the laser scanning fluorescent microscope, and may be applied to any other type of optical-beam scanning observation apparatus such as a laser scanning endoscope, which can realize a real-time observation of a living biological subject such as cells or a tissue.

The present invention enables high speed optical scanning without having detected signals interfere each other even if a plurality of beams illuminate a small region of the subject whereby high-density illuminated points are distributed thereon. Therefore, the present invention is advantageous in the case of detecting an optical signal emitted from the subject with a very low intensity, which would require long time exposure to a detecting section for the detection in a conventional scanning apparatus or method. For example, in the case when a scanning speed is increased four times higher by temporal multiplexing, the exposure time can be four times longer than that without temporal multiplexing. Furthermore, in the present invention, the apparatus needs only a single detecting device such as a photodiode (PD) or a photomultiplier tube (PMT), instead of an image device with a plurality of pixels such as a CCD or a CMOS, in order to detect signals. Furthermore, according to the present invention, the intensity of a pulsed light with temporal multiplexing can be weaker than that without temporal multiplexing in order to detect signals with a desired intensity. Therefore, an apparatus according to the present invention can be preferably used as a microscope or endoscope to image or observe a subject including fragile materials such as a living tissue, nerve cells, and the like.

### REFERENCE SIGNS LIST

1, 2, 3, 3′, 4, 5, 5′, 6, 7, 200, 201, 202, 203, 204, 206: beam splitter apparatus
8: scanning microscope
10, 20, 30, 40: optical path
11, 124: pulsed light source
12, 21, 22, 31, 32, 41, 42, 51, 52, 61, 62, 71, 72: reflection optical system (beam-angle setting section)
13, 23, 33, 63: beam splitter (branching section)
14, 25, 35, 65, 74: beam splitter (multiplexing section)
16, 17, 36, 37, 38, 39, 45, 46, 47, 48, 54, 55, 56, 57, 66, 67, 68, 69, 104, 105, 153, 161: relay optical system (pupil transfer optical system)
24, 34, 43, 44, 53, 64, 73, 154, 155: beam splitter (multiplexing/branching section)
31a, 32a: mirror (first mirror)
31b, 32b: mirror (second mirror)
31c, 32c, 51c, 52c: stage (rectilinear translation mechanism)
35′: polarizing beam splitter
49, 50: stationary mirror
83: relay lens
84, 127: dichroic mirror
85, 126: objective lens
86: detector (detecting section)
87: scanning optical system (scanning section)
88: observation optical system
101, 102, 103, 103′, 104, 105, 105′: light source apparatus
205, 207: fluoroscopy apparatus (scanning microscope)
110, 111, 112, 114, 115, 117, 118: optical fiber
113, 116, 119: fiber coupler
120: focusing lens
121, 125, 130: scanner
122, 123: positive lens
128: optical detector
129: fiber bundle

1. A beam splitter apparatus that generates a plurality of pulsed beams to be radiated on a subject from an input pulsed beam, comprising:
at least one branching section that branches the input pulsed beam into two optical paths;
at least one delaying section that endows pulsed beams passing along the two optical paths branching off via the branching section with a relative time delay to sufficiently separate responses in the subject caused by the pulsed beams; and
a beam-angle setting section that endows the plurality of pulsed beams, endowed with the relative time delay by the delaying section, with a relative angle and converges the plurality of pulsed beams on the same position.

2. The beam splitter apparatus according to claim 1, comprising:
a relay optical system that is disposed in each of the optical paths branching off via the branching section and that relays a pupil in each of the optical paths; and
at least one multiplexing section that multiplexes the plurality of pulsed beams relayed by the relay optical systems, wherein
the beam-angle setting section endows one of the pulsed beams branching off via the branching section with an angle so as to have a relative angle with respect to the other pulsed beam.

3. The beam splitter apparatus according to claim 2, wherein
the relay optical system includes at least one pair of lenses, and
the beam-angle setting section is disposed between the one pair of lenses.

4. The beam splitter apparatus according to claim 2,
wherein the beam-angle setting section includes a first mirror that reflects a pulsed beam branching off via the branching section, a second mirror that reflects the pulsed beam, reflected by the first mirror, towards the multiplexing section, and a rectilinear translation mechanism that rectilinearly translates the first mirror and the second mirror together in a direction along an optical axis therebetween.

5. The beam splitter apparatus according to claim 2,
wherein the beam-angle setting section includes a mirror that reflects the pulsed beams branching off via the branching section towards the multiplexing section, and a swing mechanism that swings the mirror about an axis orthogonal to optical axes of the pulsed beams.

6. The beam splitter apparatus according to claim 2,
wherein the beam-angle setting section includes a swing mechanism that swings at least one of the branching section and the multiplexing section about an axis orthogonal to optical axes of the pulsed beams.

7. The beam splitter apparatus according to claim 2, comprising:
a plurality of units in series that each include the branching section, the multiplexing section, the relay optical systems, and the beam-angle setting section, wherein
the beam-angle setting sections are disposed between the respective branching sections and the respective multiplexing sections.

8. The beam splitter apparatus according to claim 2, comprising:
at least one multiplexing/branching section that multiplexes the pulsed beams in the two optical paths branching off via the branching section and branches the two multiplexed pulsed beams into two optical paths with different optical path lengths,
wherein the relay optical system is disposed in each of the optical paths branching off via the branching/multiplexing section, and the beam-angle setting section endows pulsed beams branching off via the multiplexing/branching section with a relative angle.

9. The beam splitter apparatus according to claim 2, comprising:
a polarization modulator that is disposed in one of the optical paths upstream of the multiplexing section and that makes polarization states of a plurality of optical paths orthogonal to one another, wherein
the multiplexing section is a polarizing beam splitter.

10. A beam splitter apparatus that generates a plurality of pulsed beams to be radiated on a subject from an input pulsed beam, comprising:
at least one branching section that branches the input pulsed beam into two optical paths;
at least one delaying section that endows pulsed beams passing along the two optical paths branching off via the branching section with a relative time delay to sufficiently separate responses in the subject caused by the pulsed beams;
at least one multiplexing section that multiplexes the two pulsed beams endowed with the time delay by the delaying section;
a stationary displacing section that is disposed in each of the optical paths branching off via the branching section, causes pulsed beams multiplexed by the multiplexing section to be incident on different positions of the multiplexing section, and makes principal rays of the pulsed beams parallel to one another after the last multiplexing section; and
at least one focusing lens disposed after the last multiplexing section.

11. The beam splitter apparatus according to claim 10, comprising:
a relay optical system that is disposed in each of the optical paths branching off via the branching section and that relays a pupil in each of the optical paths.

12. The beam splitter apparatus according to claim 10, wherein
the stationary displacing sections include at least two mirrors and a rectilinear translation mechanism that rectilinearly translates at least one of the mirrors in a plane parallel to an optical axis of a pulsed beam incident on the mirror so as to change an optical path length between the mirrors.

13. The beam splitter apparatus according to claim 12, wherein
the rectilinear translation mechanism moves the two mirrors in a direction parallel to an optical axis between the mirrors.

14. The beam splitter apparatus according to claim 12, comprising:
downstream of the stationary displacing sections, at least one lens group and a lens-group moving mechanism that moves the lens group in a direction orthogonal to the optical axis by the same amount as an amount of displacement of the optical axis in synchronization with displacement of the optical axis by the stationary displacing section.

15. A beam splitter apparatus that generates a plurality of pulsed beams radiated on a subject from an input pulsed beam, comprising:
at least one branching section that branches the input pulsed beam into two;
at least two light-guide members with different optical path lengths that propagate the pulsed beams branching off via the branching section; and
a beam-angle setting section that endows a plurality of pulsed beams emitted from exit ends of the plurality of light-guide members with a relative angle and that converges the plurality of pulsed beams on the same position.

16. A light source apparatus comprising:
a pulsed light source that emits a pulsed beam; and
the beam splitter apparatus according to claim 1 that receives the pulsed beam emitted from the pulsed light source.

17. The light source apparatus according to claim 16, comprising:
a scanning section that spatially scans a plurality of pulsed beams emitted from the beam splitter apparatus.

18. A light source apparatus comprising:
a pulsed light source that emits a pulsed beam;
the beam splitter apparatus according to claim 15 that receives the pulsed beam emitted from the pulsed light source; and
a scanning section that spatially scans a plurality of pulsed beams emitted from the beam splitter apparatus by spatially vibrating the exit ends of the plurality of light-guide members.

19. A scanning observation apparatus comprising:
the beam splitter apparatus according to claim 1;
a scanning section that scans a plurality of pulsed beams from the beam splitter apparatus over the subject;
an observation optical system that radiates the pulsed beams scanned by the scanning section on the subject; and
a detecting section that detects the signal light collected from the subject.

20. The scanning observation apparatus according to claim 19, comprising:
a processing section that synchronizes the signal light detected by the detecting section with the scanned pulsed beams;
a restoring section that reconstructs the signal light synchronized by the processing section as two-dimensional information or three-dimensional information in association with sites on the subject; and
a display section that displays the two-dimensional information or three-dimensional information.

21. The scanning observation apparatus according to claim 19 further comprising an objective lens for focusing the beams on the subject.

22. A laser scanning microscope comprising the scanning observation apparatus according to claim 21.

23. A laser scanning endoscope comprising the scanning observation apparatus according to claim 21.

24. A light source apparatus comprising:
a pulsed light source that emits a pulsed beam; and
the beam splitter apparatus according to claim 10 that receives the pulsed beam emitted from the pulsed light source.

25. A light source apparatus comprising:
a pulsed light source that emits a pulsed beam; and
the beam splitter apparatus according to claim 15 that receives the pulsed beam emitted from the pulsed light source.

26. A scanning observation apparatus comprising:
the beam splitter apparatus according to claim 10;
a scanning section that scans a plurality of pulsed beams from the beam splitter apparatus over the subject;
an observation optical system that radiates the pulsed beams scanned by the scanning section on the subject; and
a detecting section that detects the signal light collected from the subject.

27. A scanning observation apparatus comprising:
the beam splitter apparatus according to claim 15;
a scanning section that scans a plurality of pulsed beams from the beam splitter apparatus over the subject;
an observation optical system that radiates the pulsed beams scanned by the scanning section on the subject; and
a detecting section that detects the signal light collected from the subject.
